(19) United States
(12) Patent Application Publication
(10) Pub. No.: US 2004/0220186 A1
(43) Pub. Date: Nov. 4, 2004

(54) PDE9 inhibitors for treating type 2 diabetes,metabolic syndrome, and cardiovascular disease

(75) Inventors: Andrew Simon Bell, Sandwich (GB); Michael Paul DeNinno, Gales Ferry, CT (US); Michael John Palmer, Sandwich (GB); Michael Scott Visser, Groton, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/828,485

(22) Filed: Apr. 20, 2004

Related U.S. Application Data
(60) Provisional application No. 60/466,639, filed on Apr. 30, 2003.

(51) Int. Cl. A61K 31/53; A61K 31/519; A61K 31/52; C07D 487/02; C07D 473/02

(52) U.S. Cl. 514/242; 514/260.1; 514/262.1; 514/261.1; 514/263.2; 544/184; 544/254; 544/255; 544/269

(57) ABSTRACT

The invention provides compounds of Formula (I)

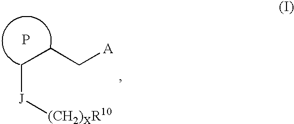

the stereoisomers and prodrugs thereof, and the pharmaceutically acceptable salts of the compounds, stereoisomers, and prodrugs, wherein A, P, J, x, and R¹⁰ are as defined herein; pharmaceutical compositions thereof; and methods of using the pharmaceutical compositions for the treatment of diseases, including, diabetes, including type 1 and type 2 diabetes, hyperglycemia, dyslipidemia, impaired glucose tolerance, metabolic syndrome, and/or cardiovascular disease.

### FIELD OF THE INVENTION

This invention relates to novel cyclic guanosine monophosphate (hereafter referred to as cGMP)-specific phosphodiesterase type 9 inhibitors (PDE9 inhibitors) for treating a variety of diseases, particularly diabetes, including type 1 and type 2 diabetes, hyperglycemia, dyslipidemia, impaired glucose tolerance, metabolic syndrome and/or cardiovascular disease. In addition, the invention relates to methods and processes concerning the same, and compositions containing the same.

### BACKGROUND OF THE INVENTION

The phosphodiesterase enzyme family hydrolyses cyclic nucleotides cGMP and cyclic adenosine monophosphate (cAMP). cGMP and cAMP are central to the control and regulation of a multitude of cellular events, both physiological and pathophysiological.

The PDE9 enzyme has been identified as a novel member of the phosphodiesterase (PDE) enzyme family that selectively hydrolyses cGMP over cAMP. See, D A Fisher et al., J. Biol. Chemistry, vol 273, No 25, 15559 -15564 (1998), which is incorporated herein by reference. PDE9 was found to be present in a variety of human tissues, namely the testes, brain, small intestine, skeletal muscle, heart, lung, thymus and spleen. We have now found the presence of PDE9 in smooth muscle cells within the human vasculature of a variety of tissues.

It is believed that insulin promotes relaxation of blood vessels at least in part through the action of nitric oxide (NO). Nitric oxide generated in the endothelium then stimulates cGMP production in blood vessels and causes them to relax or dilate. This opening of the blood vessel allows more blood to flow, which is particularly important when more blood flow is needed to critical organs, such as the heart. It has been demonstrated that there is a decreased release of NO from the endothelium of patients with insulin resistance. This decreased release of NO is not only from insulin, but also from other important vasodilators like acetylcholine. This so-called “endothelial dysfunction” contributes to the risk factors for cardiovascular disease which are associated with metabolic syndrome. The vascular effect of insulin contributes to metabolism regulation, particularly, but not necessarily limited to, glucose metabolism.

Nitric oxide also affects glucose uptake by skeletal muscle. That is, treatment with a NO-donor substance, such as nitroprusside, or with an analogue of cGMP in vitro increases glucose uptake (transport by GLUT4 glucose transporters). This vasodilation-independent pathway is described in G. J. Etgen, D. A. Fryburg and E. M. Gibbs in Diabetes, 46, 1997 pp. 1915-1919, which is incorporated herein by reference. Taken together, NO and cGMP have direct target tissue (skeletal muscle) and vascular actions that influence, mediate, or mimic the action of insulin.

Two major forms of diabetes mellitus are recognized. Type 1 diabetes, or insulin-dependent diabetes, is the result of an absolute deficiency of insulin, the hormone that regulates carbohydrate utilization. Type 2 diabetes, or non-insulin dependent diabetes, often occurs with normal, or even elevated levels of insulin and appears to be the result of the inability of tissues to respond appropriately to insulin. Complications of type 1 and 2 diabetes include retinopathy, nephropathy, neuropathy, and coronary heart disease, and are believed to be triggered by a combination of factors including excessive protein glycation and an increased flux through the polyol pathway. These abnormalities are believed to result from excessive levels of circulating glucose.

Metabolic syndrome, as defined herein, and according to the Adult Treatment Pane III (ATP III; National Institutes of Health: Third Report of the National Cholesterol Education Program Expert Panel on Detection, Evaluation, and Treatment of High Blood Cholesterol in Adults (Adult Treatment Panel III), Executive Summary; Bethesda, Md., National Institutes of Health, National Heart, Lung and Blood Institute, 2001 (NIH pub. no. 01-3670), occurs when a person has three or more of the following criteria:

1. Abdominal obesity: waist circumference >102 cm in men and >88 cm in women

2. Hypertriglyceridemia: ≧150 mg/dl (1.695 mmol/1)

3. Low HDL cholesterol: <40 mg/dl (1.036 mmol/1) in men and <50 mg/dl (1.295 mmol/l) in women

4. High blood pressure: ≧130/85 mmHg

5. High fasting glucose: ≧110 mg/dl (≧6.1 mmol/l)

Metabolic syndrome may also be linked and/or sometimes referred to as syndrome X and/or insulin resistance syndrome.

Cardiovascular disorders, diseases and/or conditions, as defined herein, include systemic (or essential) hypertension, pulmonary hypertension (e.g. pulmonary arterial hypertension, pulmonary hypertension of the neonate), congestive heart failure, coronary artery disease, atherosclerosis, stroke, thrombosis, conditions of reduced blood vessel patency (for example post percutaneous transluminal coronary angioplasty), peripheral vascular disease, renal disease (especially that occurring with diabetes), angina (including stable, unstable and variant (Prinzmetal) angina), and any condition where improved blood flow leads to improved end organ function. More preferably the cardiovascular disease is systemic hypertension.

Accordingly, there is a need for a PDE9 inhibitor that will lead to clinically relevant improvements in blood pressure, serum glucose, insulin, lipids, uric acid, and/or procoagulant factors. This treatment can occur alone or in combination with other therapeutics.

### SUMMARY OF THE INVENTION

The invention provides compounds of Formula (I),

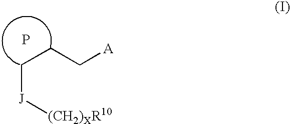

a stereoisomer or prodrug thereof, or a pharmaceutically acceptable salt of said compound, stereoisomer or prodrug thereof; wherein A, P, J, X, and R¹⁰, are as defined herein below; as well as pharmaceutical compositions thereof; and uses thereof in treating a variety of diseases, including diabetes, including type 1 and type 2 diabetes, hyperglycemia, dyslipidemia, impaired glucose tolerance, metabolic syndrome, and/or cardiovascular disease.

### DETAILED DESCRIPTION OF THE INVENTION

The invention provides compounds of Formula (I),

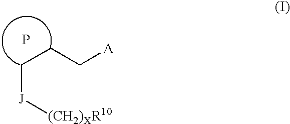

a stereoisomer or prodrug thereof, or a pharmaceutically acceptable salt of said compound, stereoisomer, or prodrug, wherein:

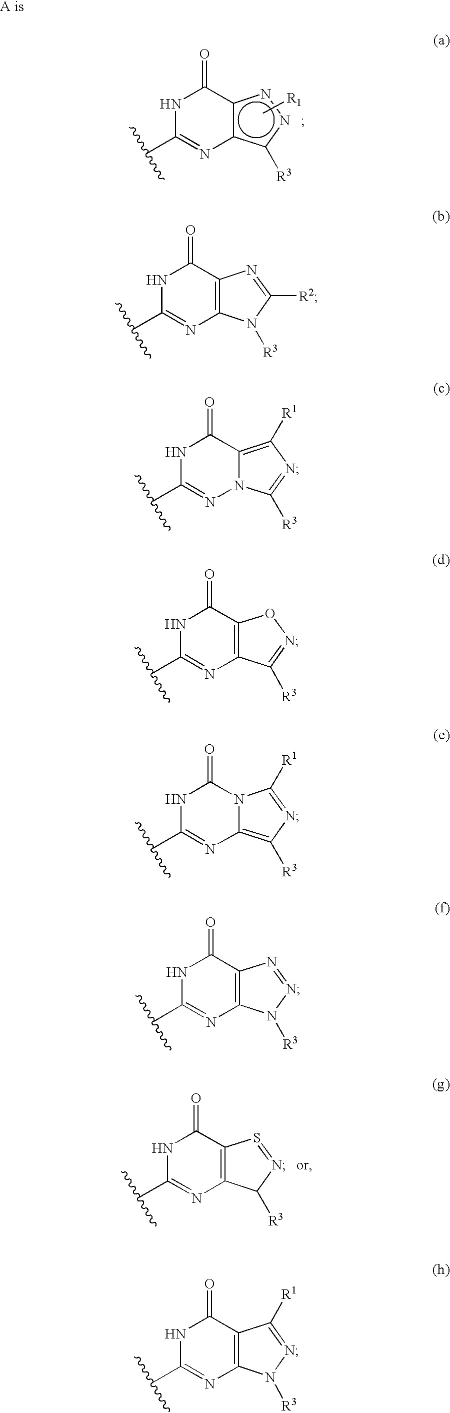

P, including the carbon atoms to which it is attached, is (C₃-C₈)cycloalkyl, (C₃-C₈)heterocycloalkyl, aryl, or heteroaryl; optionally and independently substituted with from 1 to 3 substituents independently selected from halogen, (C₁-C₅)alkyl, (C₁-C₅)alkoxy, and trifluoromethyl;

J is O, S, —N(R¹⁵)—, —N(R¹⁵)CO—, —CON(R¹⁵)—, —SO₂N(R¹⁵)—, or —N(R¹⁵)SO₂—;

x is 0, 1, 2, 3,4, 5, or 6;

R¹⁰ is —CO₂H, —CONR³⁰R³¹, —NR³⁰R³¹, or —N(R¹⁵)SO₂R⁴⁰;

R¹ and R² are independently H or (C₁-C₃)alkyl;

R³ is (C₁-C₈)alkyl, (C₃-C₈)cycloalkyl, (C₃-C₈)cycloalkyl-methyl, (C₃-C₈)heterocycloalkyl, (C₃-C₈)heterocycloalkyl-methyl, aryl, or heteroaryl; optionally and independently substituted with from 1 to 3 substituents independently selected from halogen, hydroxy, oxo, (C₁-C₅)alkyl, and (C₁-C₅)alkoxy;

R¹⁵ is H or (C₁-C₅)alkyl;

R³⁰ and R³¹ are taken separately and are independently H, (C₁-C₅)alkyl, (C₃-C₈)cycloalkyl, (C₃-C₈)heterocycloalkyl, aryl, or heteroaryl, wherein said R³⁰ and R³¹ are optionally and independently substituted with from 1 to 3 substituents independently selected from halogen, oxo, (C₁-C₅)alkyl, —CO₂R⁴⁰, —COR⁴⁰, —OR⁴⁰, —CONR⁵⁰R⁵¹, —NR⁵⁰R⁵¹, and —SO₂R⁴⁰; or

R³⁰ and R³¹ are taken together with the nitrogen atom to which they are attached to form a 5- to 8-membered heterocycloalkyl ring, said ring optionally having 1 additional heteroatom independently selected from N, O, and S, wherein said 5- to 8-membered heterocycloalkyl ring is optionally and independently substituted with from 1 to 3 substituents independently selected from halogen, oxo, (C₁-C₅)alkyl, —CO₂R⁴⁰, —COR⁴⁰, —OR⁴⁰, —CONR⁵⁰R⁵¹, —NR⁵⁰R⁵¹, and —SO₂R⁴⁰;

R⁴⁰ is H, (C₁-C₅)alkyl, (C₃-C₈)cycloalkyl, (C₃-C₈)heterocycloalkyl, aryl, or heteroaryl;

R⁵⁰ and R⁵¹ are taken separately and are independently H, (C₁-C₅)alkyl, (C₃-C₈)cycloalkyl, (C₃-C₈)heterocycloalkyl, aryl, or heteroaryl; or

R⁵⁰ and R⁵¹ are taken together with the nitrogen atom to which they are attached to form a 5- to 8-membered heterocycloalkyl ring, said ring optionally having 1 additional heteroatom independently selected from N, O, and S.

It will be appreciated by a skilled chemist that the compounds represented by general formula (I)(a) cover both compounds of (I)(a)(i) and (I)(a)(ii):

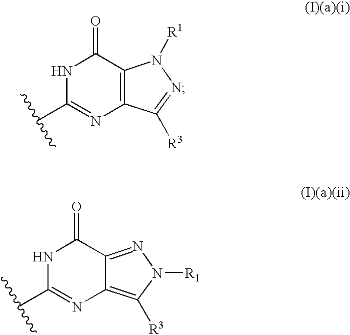

A generally preferred subgroup of the compounds of Formula (I) comprises those compounds wherein:

A is (a), (b), (c), or (h);

R¹ and R² are H;

R³ is (C₃-C₆) alkyl or (C₃-C₅) cycloalkyl;

P is (C₃-C₈)cycloalkyl or aryl;

J is O or S; and

x is 1, 2, or 3;

A more generally preferred subgroup of the compounds of Formula (I) comprises those compounds wherein:

A is (a) or (b).

The compounds and intermediates of the present invention may be named according to either the IUPAC (International Union for Pure and Applied Chemistry) or CAS (Chemical Abstracts Service, Columbus, Ohio) nomenclature systems.

The carbon atom content of the various hydrocarbon-containing moieties herein may be indicated by a prefix designating the minimum and maximum number of carbon atoms in the moiety, for example, the prefix (Cₐ-C_{b})alkyl indicates an alkyl moiety of the integer “a” to “b” carbon atoms, inclusive. Thus, for example, (C₁-C₆)alkyl refers to an alkyl group of one to six carbon atoms inclusive.

The term “alkoxy” refers to straight or branched, monovalent, saturated aliphatic chains of carbon atoms bonded to an oxygen atom. Examples of alkoxy groups include methoxy, ethoxy, propoxy, butoxy, iso-butoxy, tert-butoxy, and the like.

The term “alkyl” refers to straight or branched, monovalent, saturated aliphatic chains of carbon atoms and includes, for example, methyl, ethyl, propyl, isopropyl, butyl, isobutyl, pentyl, isopentyl, hexyl, and the like.

The term “alkenyl” denotes a straight or branched-chain hydrocarbon having one or more double bonds and includes, for example, vinyl, allyl, and the like.

The term “aryl” denotes a cyclic, aromatic hydrocarbon. Examples of aryl groups include phenyl, naphthyl, anthracenyl, phenanthrenyl, and the like.

The term “cycloalkyl” denotes a saturated monocyclic or bicyclic cycloalkyl group, optionally fused to an aromatic hydrocarbon group. Examples of cycloalkyl groups include cyclopropyl, cyclobutyl, cyclopentyl, cyclohexyl, cycloheptyl, indanyl, tetrahydronaphthalinyl, and the like.

The term “halogen” or “halo” represents chloro, bromo, fluoro, and iodo atoms and/or substituents.

The term “heteroaryl” denotes a monocyclic or polycyclic aromatic hydrocarbon group wherein one or more carbon atoms have been replaced with heteroatoms such as nitrogen, oxygen, and sulfur. If the heteroaryl group contains more than one heteroatom, the heteroatoms may be the same or different. Examples of heteroaryl groups include benzofuranyl, benzothienyl, benzimidazolyl, benzoxazolyl, benzothiazolyl, chromenyl, furyl, imidazolyl, indazolyl, indolizinyl, indolyl, isobenzofuranyl, isoindolyl, isoquinolyl, isothiazolyl, isoxazolyl, naphthyridinyl, oxadiazolyl, oxazinyl, oxazolyl, phthalazinyl, pteridinyl, purinyl, pyranyl, pyrazinyl, pyrazolyl, pyridazinyl, pyrido[3,4-b]indolyl, pyridyl, pyrimidyl, pyrrolyl, quinolizinyl, quinolyl, quinoxalinyl, thiadiazolyl, thiatriazolyl, thiazolyl, thienyl, triazinyl, triazolyl, xanthenyl, and the like.

The term “heterocycloalkyl” denotes a saturated monocyclic or polycyclic cycloalkyl group, optionally fused to an aromatic hydrocarbon group, in which at least one of the carbon atoms have been replaced with a heteroatom such as nitrogen, oxygen, and sulfur. If the heterocycloalkyl group contains more than one heteroatom, the heteroatoms may be the same or different. Examples of such heterocycloalkyl groups include azabicycloheptanyl, azetidinyl, indolinyl, morpholinyl, piperazinyl, piperidyl, pyrrolidinyl, tetrahydrofuryl, tetrahydroquinolinyl, tetrahydroindazolyl, tetrahydroindolyl, tetrahydroisoquinolinyl, tetrahydropyranyl, tetrahydroquinoxalinyl, tetrahydrothiopyranyl, thiazolidinyl, thiomorpholinyl, thioxanthenyl, thioxanyl, and the like.

A cyclic group may be bonded to another group in more than one way. If no particular bonding arrangement is specified, then all possible arrangements are intended. For example, the term “pyridyl” includes 2-, 3-, or 4-pyridyl, and the term “thienyl” includes 2- or 3-thienyl.

The term “mammal” means animals including, for example, dogs, cats, cows, sheep, horses, and humans. Preferred mammals include humans.

The term “oxo”, means a carbonyl group formed by the combination of a carbon atom(s) and an oxygen atom(s).

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “prodrug” refers to a compound that is a drug precursor which, following administration, releases the drug in vivo via a chemical or physiological process (e.g., upon being brought to physiological pH or through enzyme activity). A discussion of the synthesis and use of prodrugs is provided by T. Higuchi and W. Stella, in “Prodrugs as Novel Delivery Systems,” vol. 14 of the ACS Symposium Series, and in Bioreverible Carriers in Drug Design, ed. Edward B. Roche, American Pharmaceutical Association and Pergamon Press, 1987, both of which are incorporated herein by reference.

The term “salts” and “pharmaceutically acceptable salts” refers to organic and inorganic salts of a compound of Formula (I), or a stereoisomer, or prodrug thereof. These salts can be prepared in situ during the final isolation and purification of a compound, or by separately reacting a compound of Formula (I), or a stereoisomer, or prodrug thereof, with a suitable organic or inorganic acid or base and isolating the salt thus formed. Representative salts include the hydrobromide, hydrochloride, sulfate, bisulfate, nitrate, acetate, oxalate, besylate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may also include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. For additional examples see, for example, Berge, et al., J. Pharm. Sci., 66, 1-19 (1977), which is incorporated herein by reference.

A salt of a compound of Formula (I) may be readily prepared by mixing together solutions of a compound of Formula (I) and the desired acid or base, as appropriate. The salt may precipitate from solution and be collected by filtration or may be recovered by evaporation of the solvent.

The term “substituted” means that a hydrogen atom on a molecule has been replaced with a different atom or molecule. The atom or molecule replacing the hydrogen atom is denoted as a “substituent.”

The symbol “—” represents a covalent bond.

The phrase “reaction-inert solvent” or “inert solvent” refers to a solvent, or mixture of solvents, that does not interact with starting materials, reagents, intermediates, or products in a manner that adversely affects their desired properties.

The terms “treating”, “treated”, or “treatment” as employed herein includes preventative (e.g., prophylactic), palliative, or curative use or result.

The compounds of Formula (I) may contain asymmetric or chiral centers and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond, both the cis- and trans- forms, as well as mixtures thereof, are embraced within the scope of the invention.

Diasteriomeric mixtures can be separated into their individual diastereomers on the basis of their physical chemical differences by methods well-known to those of ordinary skill in the art, such as by chromatography and/or fractional crystallization. Enantiomers can be separated by converting the enantiomeric mixture into a diasteriomeric mixture by reaction with an appropriate optically active compound (e.g., alcohol), separating the diasteriomers and converting (e.g., hydrolyzing) the individual diasteriomers to the corresponding pure enantiomers. Also, some of the compounds of Formula (I) may be atropisomers (e.g., substituted biaryls) and are also considered as part of the invention.

The compounds of Formula (I) may exist in unsolvated as well as solvated forms with pharmaceutically acceptable solvents, such as water, ethanol, and the like, and it is intended that the invention embrace both solvated and unsolvated forms.

It is also possible that the compounds of Formula (I) may exist as tautomeric isomers in equilibrium, and all such forms are embraced within the scope of the invention.

The present invention also embraces isotopically-labeled compounds of Formula (I), which are identical to those recited herein, but for the fact that one or more atoms are replaced by an atom having an atomic mass or mass number different from the atomic mass or mass number usually found in nature. Examples of isotopes that can be incorporated into compounds of Formula (I) include isotopes of hydrogen, carbon, nitrogen, oxygen, phosphorus, fluorine, and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. The compounds of Formula (I), the stereoisomers and prodrugs thereof, and the pharmaceutically acceptable salts of the compounds, stereoisomers, or prodrugs, that contain the aforementioned isotopes and/or other isotopes of the other atoms are intended to be within the scope of the instant invention.

Certain isotopically-labeled compounds of Formula (I), for example those compounds into which radioactive isotopes such as ³H and ¹⁴C are incorporated, are useful in compound and/or substrate tissue distribution assays. Tritiated, i.e., ³H, and carbon-14, i.e., ¹⁴C, isotopes are particularly preferred for their relative ease of preparation and facile detection. Furthermore, substitution with heavier isotopes such as deuterium, i.e., ²H, may afford certain therapeutic advantages resulting from greater metabolic stability, for example, increased in vivo half-life, or reduced dosage requirements and, hence, may be preferred in some circumstances. The isotopically-labeled compounds of Formula (I) can generally be prepared by carrying out procedures analogous to those disclosed in the Schemes and/or Examples set forth hereinbelow, such as by substituting an isotopically-labeled reagent for a non-isotopically-labeled reagent.

In another aspect, the invention provides methods of treating conditions including diabetes, including type 1 and type 2 diabetes, hyperglycemia, dyslipidemia, impaired glucose tolerance, metabolic syndrome, and/or cardiovascular disease, which comprise administering to a mammal in need of such treatment, a therapeutically effective amount of a compound of Formula (I), a stereoisomer or prodrug thereof, or a pharmaceutically acceptable salt of the compound, stereoisomer, or prodrug; or a pharmaceutical composition comprising a compound of Formula (I), or a stereoisomer or prodrug thereof, or a pharmaceutically acceptable salt of the compound, stereoisomer, or prodrug, and a pharmaceutically acceptable carrier, vehicle, or diluent. A preferred condition comprises diabetes, metabolic syndrome, and/or cardiovascular disease.

In another aspect, the invention provides methods for inhibiting PDE9 activity in a mammal in need of such inhibition, which comprise administering a PDE9 inhibiting amount of a compound of Formula (I), a prodrug thereof, or a pharmaceutically acceptable salt of the compound or prodrug; or a pharmaceutical composition comprising a compound of Formula (I), a prodrug thereof, or a pharmaceutically acceptable salt of the compound or prodrug, and a pharmaceutically acceptable carrier, vehicle, or diluent.

The compounds of Formula (I) may be administered to a mammal at dosage levels in the range of from about 0.1 mg to about 3,000 mg per day. For a normal adult human having a body mass of about 70 kg, a dosage in the range of from about 0.01 mg to about 100 mg per kg body mass is typically sufficient, and preferably from about 0.1 mg to about 10 mg per kg. However, some variability in the general dosage range may be required depending upon the age and mass of the subject being treated, the intended route of administration, the particular compound being administered, and the like. The determination of dosage ranges and optimal dosages for a particular mammalian subject is within the ability of one of ordinary skill in the art having benefit of the instant disclosure.

According to the methods of the present invention, a compound of Formula (I), a stereoisomer or prodrug thereof, or a pharmaceutically acceptable salt of the compound, stereoisomer, or prodrug, may be administered in the form of a pharmaceutical composition comprising a pharmaceutically acceptable carrier, vehicle, or diluent. Accordingly, a compound of Formula (I), a stereoisomer or prodrug thereof, or a pharmaceutically acceptable salt of the compound, stereoisomer, or prodrug, may be administered to a subject separately or together in any conventional dosage forms, including, oral, buccal, sublingual, ocular, topical (e.g., transdermal), parenteral (e.g., intravenous, intramuscular, or subcutaneous), rectal, intracisternal, intravaginal, intraperitoneal, intravesical, local (e.g., powder, ointment, or drop), nasal and/or inhalation dosage forms.

Pharmaceutical compositions suitable for parenteral injection may comprise pharmaceutically acceptable sterile aqueous or nonaqueous solutions, dispersions, suspensions, or emulsions, and sterile powders for extemporaneous reconstitution into sterile injectable solutions or dispersions. Examples of suitable aqueous and nonaqueous carriers, vehicles, and diluents include water, ethanol, polyols (such as propylene glycol, polyethylene glycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions, and by the use of surfactants.

The pharmaceutical compositions of the invention may further comprise adjuvants, such as preserving, wetting, emulsifying, and dispersing agents. Prevention of microorganism contamination of the instant compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions may be affected by the use of agents capable of delaying absorption, for example, aluminum monostearate and gelatin.

Solid dosage forms for oral administration include capsules, tablets, powders, and granules. In such solid dosage forms, the active compound is admixed with at least one inert conventional pharmaceutical excipient (or carrier) such as sodium citrate or dicalcium phosphate, or (a) fillers or extenders, such as for example, starches, lactose, sucrose, mannitol, and silicic acid; (b) binders, such as for example, carboxymethylcellulose, alginates, gelatin, polyvinylpyrrolidone, sucrose, and acacia; (c) humectants, such as for example, glycerol; (d) disintegrating agents, such as for example, agar-agar, calcium carbonate, potato or tapioca starch, alginic acid certain complex silicates, and sodium carbonate; (e) solution retarders, such as for example, paraffin; (f) absorption accelerators, such as for example, quaternary ammonium compounds; (g) wetting agents, such as for example, cetyl alcohol and glycerol monostearate; (h) adsorbents, such as for example, kaolin and bentonite; and/or (i) lubricants, such as for example, talc, calcium stearate, magnesium stearate, solid polyethylene glycols, sodium lauryl sulfate, or mixtures thereof. In the case of capsules and tablets, the dosage forms may further comprise buffering agents.

Solid dosage forms may be formulated as modified release and pulsatile release dosage forms containing excipients such as those detailed above for immediate release dosage forms together with additional excipients that act as release rate modifiers, these being coated on and/or included in the body of the device. Release rate modifiers include, but are not limited to, hydroxypropylmethyl cellulose, methyl cellulose, sodium carboxymethylcellulose, ethyl cellulose, cellulose acetate, polyethylene oxide, Xanthan gum, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients.

The pharmaceutical compositions of the invention may further comprise fast dispersing or dissolving dosage formulations (FDDFs) containing the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e., where the drug substance is insoluble, a fast dispersing dosage form may be prepared, and where the drug substance is soluble, a fast dissolving dosage form may be prepared.

Solid compositions of a similar type may also be employed as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings and shells, such as enteric coatings and others well-known to one of ordinary skill in the art. They may also comprise opacifying agents, and can also be of such composition that they release the active compound(s) in a delayed, sustained, or controlled manner. Examples of embedding compositions that can be employed are polymeric substances and waxes. The active compound(s) can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, benzyl benzoate, propylene glycol, 1,3-butylene glycol, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the pharmaceutical composition can also include adjuvants, such as wetting agents, emulsifying and suspending agents, sweetening, flavoring, and perfuming agents.

In addition to the active compound(s), the pharmaceutical composition may further include suspending agents, such as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Pharmaceutical compositions of the present invention may also be configured for treatments in veterinary use, where a compound of the present invention, or a veterinarily acceptable salt thereof, or veterinarily acceptable solvate or pro-drug thereof, is administered as a suitably acceptable formulation in accordance with normal veterinary practice and the veterinary practitioner will determine the dosing regimen and route of administration which will be most appropriate for a particular animal.

The present invention additionally comprises the combination of a PDE9 inhibitor compound as provided in Formula (I) and one or more additional antidiabetic and/or cardiovascular agent(s).

The present invention additionally comprises the combination of a PDE9 inhibitor, such as provided in Formula (I), and one or more additional active agent selected from:

a) a PGI2 prostaglandin, such as prostacyclin or iloprost;

b) an α-adrenergic receptor antagonist compound also known as αadrenoceptor antagonists, α-receptor antagonists or α-blockers; suitable compounds for use herein include: the α-adrenergic receptor antagonists as described in PCT application WO99/30697 published on 14th Jun. 1998, the disclosures of which relating to α-adrenergic receptor antagonists are incorporated herein by reference and include, selective α₁-adrenoceptor antagonists or α₂-adrenoceptor antagonists and non-selective adrenoceptor antagonists, suitable α₁-adrenoceptor antagonists include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, doxazosin, terazosin, abanoquil and prazosin; α₂-blockers from U.S. Pat. No. 6,037,346, dibenarnine, tolazoline, trimazosin and dibenarnine; α-adrenergic receptors as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-adrenoceptor antagonists include: clonidine, papaverine, papaverine hydrochloride, optionally in the presence of a cariotonic agent such as pirxamine;

c) an NO-donor (NO-agonist) compound; suitable NO-donor compounds for use herein include organic nitrates, such as mono- di or tri-nitrates or organic nitrate esters including glyceryl trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP), S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates, (NONOates), 1,5-pentanedinitrate, L-arginene, ginseng, zizphi fructus, molsidomine, and nitrosylated maxisylyte derivatives such as NMI-678-11 and NMI-937 as described in published PCT application WO 0012075;

d) a potassium channel opener; suitable potassium channel openers for use herein include nicorandil, cromokalim, levcromakalim, lemakalim, pinacidil, cliazoxide, minoxidil, charybdotoxin, glyburide, 4-aminopyridine, and barium chloride;

e) a compound which modulates the action of atrial natruretic factor (also known as atrial naturetic peptide), such as inhibitors of neutral endopeptidase (NEP);

f) a compound which inhibits angiotensin-converting enzyme (ACE), such as alacepril, alindapril, altiopril, benazepril, benazeprilat, captopril, ceronapril, cilazapril, cilazaprilat, delapril, enalapril, enalaprilat, fosinopril, imidapril, indolapril, libenzapril, lisinopril, moexepril, moveltipril, pentopril, perindopril, quinapril, quinaprilat, ramipril, rentiapril, spirapril, temocapril, teprotide, trandolapril and zofenopril, or a dual ACE/NEP inhibitor, i.e. a compound that inhibits both ACE and NEP, such as, for example, omapatrilat, fasidotril, and mixanpril;

g) an angiotensin II receptor blocker (ARB) such as candesartan, eprosartan, irbesartan, losartan, olmesartan, olmesartan medoxomil, saralasin, telmisartan and valsartan;

h) a substrate for NO-synthase, such as L-arginine;

i) a calcium channel blocker such as amlodipine, verapamil, pranidipine, azelnidipine and vatanidipine;

j) an antagonist of endothelin receptors or an inhibitor of endothelin-converting enzyme;

k) a cholesterol lowering agent such as statins, such as, for example, atorvastatin calcium (Lipitor), cerivastatin sodium (Baycol), fluvastatin sodium (Lescol), lovastatin (Mevacor), pravastatin sodium (Pravachol), and simvastatin (Zocor);

l) an antiplatelet or antithrombotic agent, e.g. tPA, uPA, warfarin, hirudin and other thrombin inhibitors, aspirin, plavix, cilastozol, heparin, and thromboplastin activating factor inhibitors;

m) a PDE5 inhibitor (such as 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil); (6R, 12aR)-2,3,6,7,12,12a-hexahydro-2-methyl-6-(3,4-methylenedioxyphenyl)pyrazino[2′,1′:6,1]pyrido[3,4-b]indole-1,4-dione (tadalafil, IC-351); 2-[2-ethoxy-5-(4-ethyl-piperazin-1-yl-1-sulphonyl)-phenyl]-5-methyl-7-propyl-3H-imidazo[5,1-][1,2,4]triazin-4-one (vardenafil); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; and 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one); and

n) a beta-blocker, diuretic or aldosterone antagonist.

If a combination of active agents is administered, then they may be administered simultaneously, separately or sequentially.

Compounds of Formula (I) may be prepared by the following reaction exemplary synthetic routes (“schemes”), as well as by other conventional organic preparative methods. These processes form further aspects of the invention. General formulae are designated by Roman numerals I, II, III, IV etc. Subsets of these general formulae are designated Ia, Ib, Ic, etc. . . . IVa, IVb, IVc, etc. For example, it is to be understood that reference to Formula “Ia” refers to the compound depicted by Formula (I) together with a ring structure selected from the ring structures identified in Group A; in this example, structure “(a).” Likewise, it is also understood that reference to Formula IIm or IIIm refers to the compounds depicted by Formulas II or III, together with a ring structure selected from the ring structures identified in Group Q; in this example, structure “(m).” It is further understood that these methods are intended for purposes of exemplifying the instant invention only, and are not to be construed in any manner as limitations thereon.

Target Formula (I) compounds may contain primary or secondary amine groups or carboxylic acids in protected form that require deprotection as the last step. The use of such protecting groups is well known to those skilled in the art. For a detailed reference on the use of protecting groups see: T. A. Greene, P. G. M. Wuts “Protecting Groups in Organic Synthesis,” Vol. 2. Wiley and Sons, 1991, which is incorporated herein by reference.

Compounds of general Formula (I), wherein A is (a), (b), (d), (f), (g), or (h), may be prepared from compounds of general Formula II according to Scheme 1. Suitable conditions are well known to those skilled in the art and include a base catalyzed cyclization using reagents such as potassium tert-butoxide, sodium hydroxide and potassium carbonate in an alcoholic solvent such as ethanol or isopropanol or an alcohol/water mixture. The reaction may be carried out at a temperature between ambient temperature and the reflux temperature of the solvent, and optionally in the presence of hydrogen peroxide.

Alternatively, compounds of Formula (I), wherein A is (a), (b), (d), (f), (g), or (h), may be constructed by condensation of Formula III compounds with esters IVb under base catalysis. Typically, the Formula III and IVb compounds are treated with a base, such as potassium t-butoxide in a protic solvent such as 1-butanol at elevated temperatures of about 80° C. to about 120° C. for about two hours to about 24 hours. The reaction can also be heated in a microwave apparatus. Typically, the reaction is heated at a temperature of about 150° C. to about 200° C., preferably at about 180° C. for about five minutes to about 30 minutes.

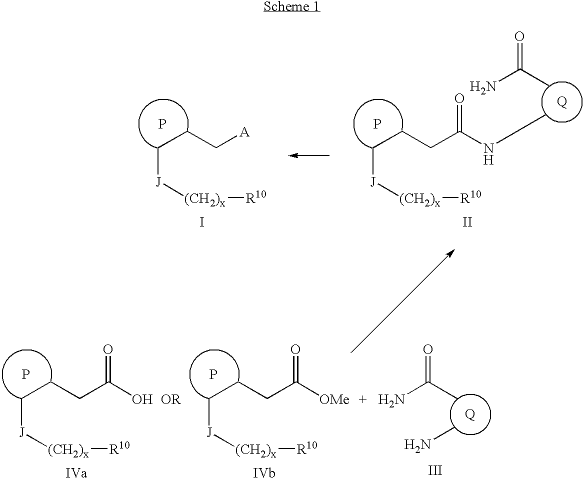

Compounds of Formula II may be prepared by reacting compounds of Formula IVa with compounds of Formula III. Such amide bond forming reactions may be carried out under a wide variety of conditions well known to those skilled in the art. For example, compounds of Formula IVa may be activated by treatment with an agent such as 1,1-carbonyldiimidazole (CDI) or fluoro-N,N,N′,N′-tetramethylformamidinium hexafluorophosphate (TFFH), or a combination of agents such as azabenzotriazol-1-yloxytris(pyrrolidino)-phosphonium hexafluorophosphate (PyAOP) and 1-hydroxy-7-azabenzotriazole (HOAt), followed by addition of the compound of Formula III.

Q is selected from

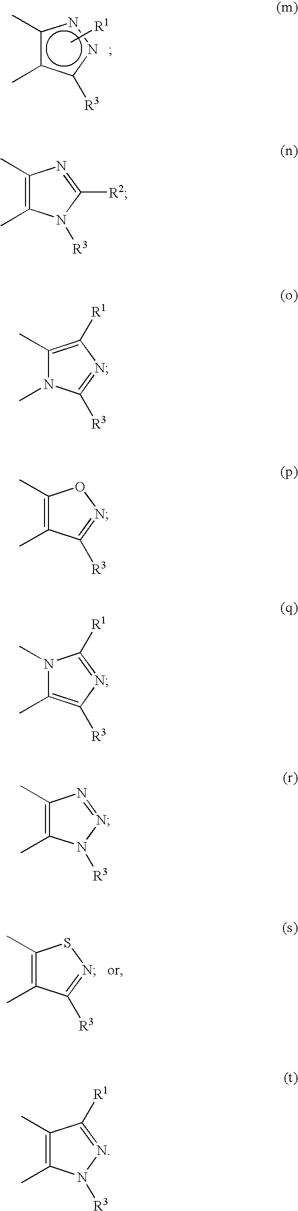

Alternatively, Formula II compounds may be prepared by addition of a peptide coupling agent such as O-(7-azabenzotriazol-1-yl)-N,N,N′,N′-tetramethyluronium hexafluorophosphate (HATU) or 1-propanephosphonic acid anhydride (T3P) to a mixture of the compounds of Formula III and IVa. This reaction is carried out in a suitable solvent such as dichloromethane, pyridine, N,N-dimethylformamide (DMF), N,N-dimethylacetamide (DMA) or 1-methyl-2-pyrrolidinone at a temperature between 0° C. and the reflux temperature of the solvent. The reaction is preferentially carried out by activation of the compound of Formula IVa with T3P and triethylamine at ambient temperatures.

Compounds of Formula III(n), wherein R² and R³ are defined above, can be prepared from an appropriate amine and Formula V compound (Scheme 2). The Formula V compound is first treated with a trialkyl ortho ester, preferably triethyl ortho formate, in a non-protic solvent, such as acetonitrile at a temperature of about 50° C. to about 80° C. for about 0.5 hours to about three hours. The R³—NH₂ Formula VII compound is then added and the reaction cooled and stirred at ambient temperature for about 12 hours to about 24 hours.

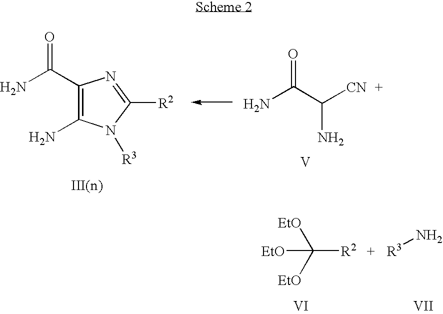

Compounds of Formula III(m), wherein R¹ and R³ are defined above, can be prepared from the corresponding Formula VIII nitro compounds by a reduction (Scheme 3). Typically the reduction is performed by catalytic hydrogenation. The Formula VIII compound is treated with a noble metal catalyst, preferably Pd/C, in a reaction-inert solvent, such as ethanol under hydrogen atmosphere of 15-45 psi at ambient temperature for about one hour to about six hours.

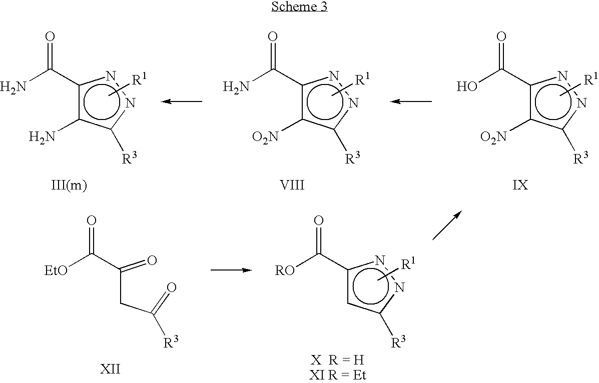

Compounds of Formula VIII, wherein R¹ and R³ are defined above, are prepared from the appropriate Formula IX compound via an amide forming reaction. Generally, the Formula IX compound is first converted to the acid chloride by reaction with oxalyl chloride in a reaction-inert solvent, such as methylene chloride, containing a catalytic amount of DMF at ambient temperature for about four hours to about 24 hours. The intermediate acid chloride is then treated with excess ammonia in a suitable solvent, such as tetrahydrofuran or dioxane, or mixtures thereof, at about 0° C. to about ambient temperature for about one hour to about 24 hours.

Compounds of Formula IX, wherein R¹ and R³ are defined above, may be prepared by nitrating a Formula X compound. Typically the Formula X compound is dissolved in sulfuric acid and treated with nitric acid at elevated temperatures of about 40° C. to about 80° C., preferably at about 60° C. for about one hour to about six hours.

Compounds of Formula X, wherein R¹ and R³ are defined above, may be prepared by saponifying a Formula XI compound. Typically the Formula XI compound is treated with an alkali base, such as sodium hydroxide, in a protic solvent, such as methanol or ethanol at a temperature of about 30° C. to about 80° C. for one hour to about six hours.

Compounds of Formula XI, wherein R¹ and R³ are defined above, may be prepared by reacting a Formula XII compound with a hydrazine. Typically the Formula XII compound is treated with hydrazine in a protic solvent, such as ethanol, at ambient temperature for about 10 hours to about 24 hours.

Compounds of Formula III(p), wherein R³ is defined above, may be prepared by reducing a Formula XIII compound (Scheme 4). Generally the reduction is performed by treating the Formula XIII nitro compound with a powdered transition metal, preferably zinc, and a proton source such as acetic acid or ammonium chloride in a protic solvent such as water at ambient temperature for about 30 minutes to about three hours.

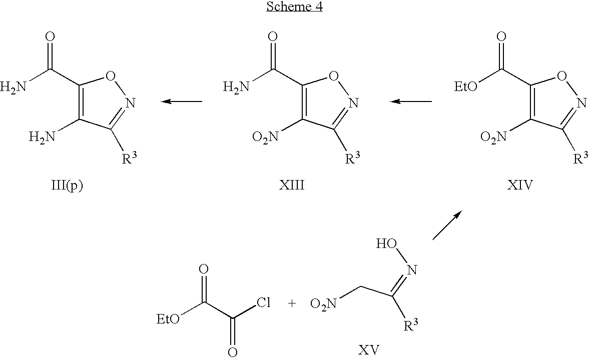

Compounds of Formula XIII, wherein R³ is defined above, may be prepared from the corresponding Formula XIV ester compound through an amidation reaction. Typically, the ester is treated with excess ammonia in a reaction inert solvent such as ethanol or water at ambient temperature for about two hours to about 24 hours.

Compounds of Formula XIV, wherein R³ is defined above, may be prepared by reaction of the appropriate Formula XV compound with ethyl oxalyl chloride. Generally, the reaction is carried out in ethereal solvent such as diethyl ether or tetrahydrofuran, and catalyzed by a base, preferably an amine base, most preferably triethylamine, at a temperature of about 0° C. to about ambient temperature for about one hour to about four hours.

Compounds of Formula III(r), wherein R³ is defined above, may be prepared from the cyano amide XVI and azide XVII (Scheme 5). Generally, the Formula XVI and Formula XVII compounds are added to a solution of sodium ethoxide in ethanol at an elevated temperature of about 40° C. to about 80° C. for about 30 minutes to about four hours.

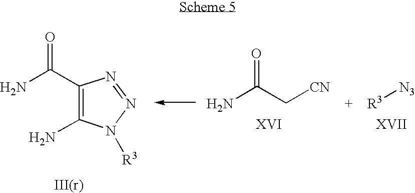

Compounds of Formula III(t), wherein R¹ and R³ are defined above, may be prepared from the corresponding Formula XVIII compounds by a hydrolysis reaction (Scheme 6). Typically, the Formula XVIII compound is treated with an acid, preferably sulfuric acid, at a temperature of about 10° C. to about 20° C., for about two hours to about four hours.

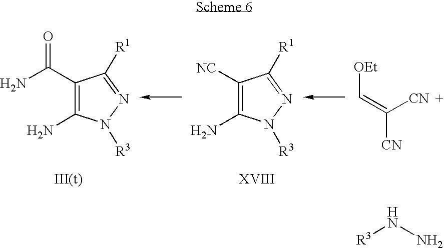

Compounds of Formula XVIII are prepared from the corresponding Formula XIX and Formula XX compounds. Generally, the Formula XIX compound is added slowly to a solution of Formula XX compound in an alcohol solvent, preferably ethanol, at an elevated temperature, preferably at reflux temperature for about one hour to about three hours.

Compounds of Formula III(s), wherein R³ is defined above, can be prepared from the corresponding Formula XXI and Formula XXII compounds (Scheme 7). Generally, a mixture of Formula XXI compound and Formula XXII compound in an alcohol solvent, such as methanol, is treated with an amine base, preferably triethyl amine, at a temperature of about ambient temperature to about 50° C. for about one hour to about four hours.

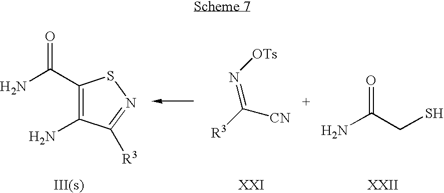

Compounds of Formula Ic, wherein R¹, R³, P, J, x, and R¹⁰ are defined above, can be prepared by condensing the corresponding Formula XXIII and Formula XXIV compounds (Scheme 8). Typically the compounds are dissolved in a protic solvent, preferably methanol, at elevated temperature, preferably at reflux temperature, for about two hours to about eight hours. The crude product is then cyclized by reaction with a chlorinating agent, such as phosphorus oxychloride in a halogenated solvent such as dichloro ethane at a temperature of about 50° C. to about 90° C. for about one hour to about four hours.

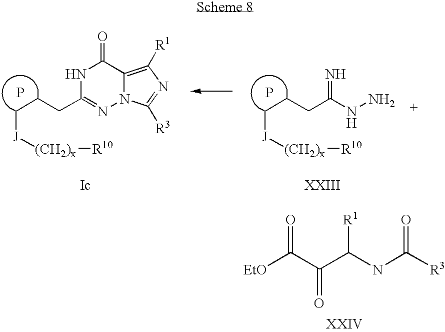

Compounds of Formula Ie, wherein R¹, R³, P, J, x, and R¹⁰ are defined above, may be prepared from the corresponding Formula XXV compounds (Scheme 9). Generally the Formula XXV compound is treated with trimethylsilyl chloride in an amine base used as solvent, preferably pyridine, at ambient temperature for about 15 minutes to about 30 minutes. Hexamethyldisilazane is then added and the mixture heated at a temperature of about 80° C. to about 120° C., preferably at reflux temperature of the solvent for about six hours to about 24 hours.

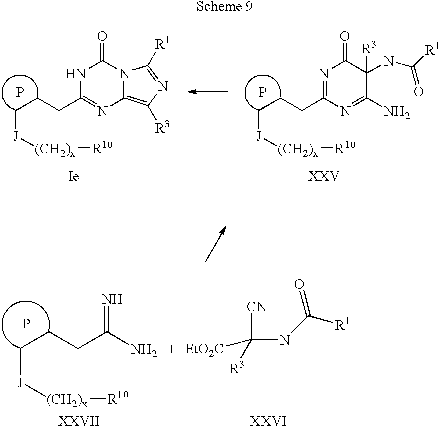

The desired Formula XXV compounds can be prepared by condensation of the corresponding Formula XXVI and XXVII compounds. The reaction is catalyzed by a base, such as sodium ethoxide, in an alcohol solvent, such as ethanol at elevated temperature, preferably at reflux temperature of the solvent for about one hour to about eight hours.

The various carboxylic acid and ester Formula IV compounds can be prepared readily from commercially available starting materials by methods known to those skilled in the art. The following general procedures are given as examples of some of these methods (Scheme 10).

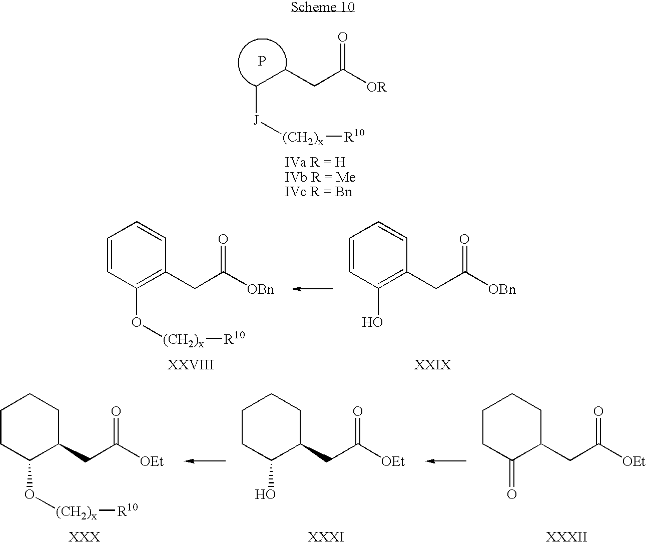

Compounds of Formula IVa, wherein J, x, and R¹⁰ are defined above, can be prepared from the corresponding Formula IVb ester compounds by a saponification reaction. The Formula IVb compound may be treated with an alkali base, such as sodium hydroxide, in a protic solvent, such as methanol or ethanol at a temperature of about 30° C. to about 80° C. for one hour to about six hours.

Alternatively, Formula IVa compounds may be prepared by deprotecting the corresponding benzyl esters (Formula IVc compounds). This is generally accomplished by catalytic hydrogenolysis. The Formula IVc compound is treated with a noble metal catalyst, preferably Pd/C, in a reaction-inert solvent, such as ethanol under hydrogen atmosphere of 15-45 psi at ambient temperature for about one hour to about six hours.

Compounds of Formula XXVIII may be prepared by alkylation of a Formula XXIX compound. Thus the Formula XXVIII compound is treated with R¹⁰(CH₂)ₓL wherein L is a leaving group under base catalysis, such as potassium carbonate in a polar, aprotic solvent such as dimethyl formamide at a temperature of ambient temperature to about 50° C. for about four hours to about 24 hours. Alternatively the alkylation can be accomplished via Mitsunobu conditions. Generally, the Formula XXVIII compound is treated with an alcohol such as R¹⁰(CH₂)ₓOH, triphenyl phosphine, and diethylazodicarboxylate in a reaction inert solvent such as tetrahydrofuran at ambient temperature for about six hours to about 24 hours.

Similarly, Formula XXX compounds, wherein R¹⁰ and x are defined above, can be prepared from the corresponding Formula XXXI alcohol compounds by an alkylation reaction. Generally the Formula XXXI compound is treated with R¹⁰(CH₂)ₓL wherein L is a leaving group under base catalysis, such as sodium hydride in a polar, aprotic solvent such as dimethyl formamide at a temperature of about 0° C. to about ambient temperature for about four hours to about 24 hours. The Formula XXXI compound may be prepared from the corresponding ketone Formula XXXVII compound by a reduction. Typically the reduction is carried out by treatment of the ketone with a reducing agent, such as sodium borohydride, in an alcohol solvent, preferably ethanol, at ambient temperature for about 30 minutes to about four hours.

Compounds described in Examples 1-30, were subject to liquid chromatography mass spectroscopy (LC-S) under the conditions described below.

LC-MS Conditions

The molecular weight and retention time determinations for the examples were performed on a reverse phase LCMS system. The column was a Polaris C18-A, 5 μm, 20×2.0 mm run at ambient temperature. The compounds were eluted with a gradient solvent system. Eluent A was 0.05% Formic Acid; 98% Water (HPLC); 2% Acetonitrile and eluent B was 0.005% Formic Acid in Acetonitrile., The initial pump conditions were A % 95, B % 5 with a flow rate of 1 ml/min at a pressure of 270 bar. The solvent gradient went from 5% to 95% eluent B over 3.74 minutes. Products were detected by both UV, and MS APCI methods. UV data was obtained on a Hewlett Packard 1100 DAD (Hewlett Packard, Palo Alto, Calif.). MS data was obtained on a Micromass ZMD (Micromass, Inc., Manchester, UK). NMR data was obtained on a Varian Unity 400 (Varian, Inc., Palo Alto, Calif.).

### EXAMPLES

The Examples set forth hereinbelow are for illustrative purposes only. The compositions, methods, and various parameters reflected therein are intended only to exemplify various aspects and embodiments of the invention, and are not intended to limit the scope of the claimed invention in any way.

Unless noted otherwise, all reactants were obtained commercially. Unless indicated otherwise, the following experimental abbreviations have the indicated meanings:

BOC—t-butyl oxycarbonyl

DMAP—dimethyl aminopyridine

DMF—dimethylformamide

DMSO—dimethyl sulfoxide

ES/MS—electron spray mass spectrometry

EtOAc—ethyl acetate

EtOH—ethanol

H (e.g., 1H, 2H)—hydrogen(s)

h (e.g., 1 h, 2 h)—hour(s)

LC—liquid chromatography

(M-BOC)—mass—BOC

MeOH—methanol

min(s)—minute(s)

MS—mass spectroscopy

NMR—nuclear magnetic resonance

RT—room temperature

THF—tetrahydrofuran

### Example 1

1-{[2-(3-Isopropyl-7-oxo-6,7-dihydro-1H-pyrazolo[4,3-d]pyrimidin-5-ylmethyl)-phenoxy}-acetyl]-pyrrolidine-2-carboxylic acid

Sodium hydroxide (0.5 mL of a 15% solution) was added to a solution of 1-{[2-(3-isopropyl-7-oxo-6,7-dihydro-1H-pyrazolo[4,3-d]pyrimidin-5-ylmethyl)-phenoxy]-acetyl}-pyrrolidine-2-carboxylic acid ethyl ester (81 mg, 0.18 mmol) in methanol (10 mL) at RT. The mixture was heated at 58° C. for 2 h, then cooled, acidified with 1N HCl, and extracted with EtOAc (2×). The combined extracts were dried (MgSO₄), filtered and concentrated to afford 40 mg of the title compound as a colorless solid.

MS 440 (M+H)⁺; ¹H NMR (400 MHz, CD₃OD) δ 7.2 (m, 2H); 6.95 (m, 2H); 4.6 (m, 2H); 4.04 (m, 2H); 3.6 (m, 4h); 3.4 (m, 1H); 2.3-1.9 (m, 3H); 1.38 (d, 6H, J=7.1 Hz). C₂₂H₂₅N₅O₅. LC retention time: 1.7 min.

The following compound, Example 2, was prepared from the appropriate ester according to the general procedure above for the preparation of Example 1.

### Example 2

1-{[2-(1-Cyclopentyl-4-oxo-4,5-dihydro-1H-pyrazolo[3,4-d]pyrimidin-6-ylmethyl)-phenoxy]-acetyl}-pyrrolidine-2(S)-carboxylic acid C₂₄H₂₇N₅O₅. MS 466 (M+H)⁺. LC retention time: 2.0 min.

### Example 3

3-Isopropyl-5-[2-(2-oxo-2-piperazin-1-yl-ethoxy)-benzyl]-1,6-dihydro-pyrazolo[4,3-d]pyrimidin-7-one trifluoro acetate.

Trifluoroacetic acid (3 mL) was added to 4-{[2-(3-Isopropyl-7-oxo-6,7-dihydro-1H-pyrazolo[4,3-d]pyrimidin-5-ylmethyl)-phenoxy]-acetyl}-piperazine-1-carboxylic acid tert-butyl ester (80 mg, 0.16 mmol) and the mixture stirred at RT for 2 h. The mixture was concentrated in vacuo and then reconcentrated from toluene 3×. The residue was crystallized from methanol/ether to afford 62 mg of the title compound as a colorless solid.

MS 411 (M+H)⁺; ¹H NMR (400 MHz, CD₃OD) δ 7.23 (m, 2H); 6.98 (m, 2H); 5.0 (s, 2H); 4.06 (s, 2H); 3.90 (bs, 2H); 3.80 (bs, 2H); 3.4 (m, 1H); 3.25 (m, 4H); 1.39 (d, 6H, J=7.0 Hz). C₂₁H₂₆N₆O₃C₂HF₃O₂. LC retention time: 1.3 min.

The following compound, Example 4, was prepared from the appropriate BOC-protected amine according to the general procedure above for the preparation of Example 3.

### Example 4

1-Cyclopentyl-6-[2-(2-oxo-2-piperazin-1-yl-ethoxy)-benzyl]-1.5-dihydro-pyrazolo[3,4-d]pyrimidin-4-one C₂₃H₂₈N₆O₃. MS 437 (M+H)⁺. LC retention time: 1.6 min.

### Example 5

3-Isopropyl-5-[2-(2-morpholin-4-yl-2-oxo-ethoxy)-benzyl]-1,6-dihydro-pyrazolo[4,3-d]pyrimidin-7-one

1-Propanephosphonic acid cyclic anhydride (50% solution in ethyl acetate, 0.1 mL, 0.175 mmol) was added to a solution of [2-(3-Isopropyl-7-oxo-6,7-dihydro-1H-pyrazolo[4,3-d]pyrimidin-5-ylmethyl)-phenoxy]-acetic acid (50 mg, 0.15 mmol) and morpholine (50 uL, 0.58 mmol) in anhydrous DMF (3 mL) at RT. After 20 h, the mixture was concentrated in vacuo, and the residue purified by flash chromatography (1→2→4→5% MeOH/CH₂Cl₂ with 0.2% NH₄OH) afforded 38 mg of the title compound as a colorless solid.

MS 412 (M+H)⁺; ¹H NMR (400 MHz, CD₃OD) δ 7.21 (m, 2H); 6.97 (m, 2H); 4.93 (s, 2H); 4.03 (bs, 2H); 3.62 (m, 6H); 3.5 (m, 2H); 3.4 (m, 1H); 1.4 (m, 6H). C₂₁H₂₅N₅O₄. LC retention time: 1.8 min.

The following compounds, Examples 6-18, were prepared from the appropriate amine according to the general procedure above for the preparation of Example 5.

### Example 6

3-Isopropyl-5-[2-(2-oxo-2-pyrrolidin-1-yl-ethoxy)-benzyl]-1,6-dihydro-pyrazolo[4,3-d]pyrimidin-7-one. C₂₁H₂₅N₅O₃. MS 396 (M+H)⁺. LC retention time: 1.9 min.

### Example 7

5-{2-[2-(4-Ethyl-piperazin-1-yl)-2-oxo-ethoxy}-benzyl]-3-isopropyl-1,6-dihydro-pyrazolo[4,3-d]pyrimidin-7-one. C₂₃H₃₀N₆O₃. MS 439 (M+H)⁺. LC retention time: 1.3 min.

### Example 8

N,N-Diethyl-2-[2-(3-isopropyl-7-oxo-6,7-dihydro-1H-pyrazolo[4,3-d]pyrimidin-5-ylmethyl)-phenoxy]-acetamide. C₂₁H₂₇N₅O₃. MS 398 (M+H)⁺. LC retention time: 2.0 min.

### Example 9

1-{[2-(3-Isopropyl-7-oxo-6,7-dihydro-1H-pyrazolo[4,3-d]pyrimidin-5-ylmethyl)-phenoxy]-acetyl}-pyrrolidine-2-carboxylic acid methyl ester. C₂₃H₂₇N₅O₅. MS 454 (M+H)⁺. LC retention time: 1.9 min.

### Example 10

4-{[2-(3-Isopropyl-7-oxo-6,7-dihydro-1H-pyrazolo[4,3-d]pyrimidin-5-ylmethyl)-phenoxy]-acetyl}-piperazine-1-carboxylic acid tert-butyl ester. C₂₆H₃₄N₆O₅. MS 411 (M−BOC)⁺. LC retention time: 2.3 min.

### Example 11

N-(2-Dimethylamino-ethyl)-2-[2-(3-isopropyl-7-oxo-6,7-dihydro-1H-Pyrazolo[4,3-d]pyrimidin-5-ylmethyl)-phenoxy]-acetamide. C₂₁H₂₈N₆O₃. MS 413 (M+H)⁺. LC retention time: 1.2 min.

### Example 12

1-{[2-(1-Cyclopentyl-4-oxo-4,5-dihydro-1H-pyrazolo[3,4-d]pyrimidin-6-ylmethyl)-phenoxy]-acetyl}-pyrrolidine-2-carboxylic acid methyl ester C₂₅H₂₉N₅O₅. MS 480 (M+H)⁺. LC retention time: 2.3 min.

### Example 13

4-{[2-(1-Cyclopentyl-4-oxo-4,5-dihydro-1H-pyrazolo[3,4-d]pyrimidin-6-ylmethyl)-phenoxy]-acetyl}-piperazine-1-carboxylic acid tert-butyl ester C₂₈H₃₆N₆O₅. MS 537 (M+H)⁺. LC retention time: 2.6 min.

### Example 14

1-Cyclopentyl-6-[2-(2-oxo-2-pyrrolidin-1-yl-ethoxy)-benzyl]-1,5-dihydro-pyrazolo[3,4-d]pyrimidin-4-one C₂₃H₂₇N₅O₃. MS 422 (M+H)⁺. LC retention time: 2.9 min.

### Example 15

1-Cyclopentyl-6-[2-(2-morpholin-4-yl-2-oxo-ethoxy)-benzyl]-1,5-dihydro-pyrazolo[3,4-d]pyrimidin-4-one C₂₃H₂₇N₅O₄. MS 438 (M+H)⁺. LC retention time: 2.8 min.

### Example 16

2-[2-(1-Cyclopentyl-4-oxo-4,5-dihydro-1H-pyrazolo[3,4-d]pyrimidin-6-ylmethyl)-phenoxy]-N-(2-dimethylamino-ethyl)-acetamide C₂₈H₃₀N₆O₃. MS 439 (M+H)⁺. LC retention time: 1.8 min.

### Example 17

1-Cyclopentyl-6-{2-[2-(4-ethyl-piperazin-1-yl)-2-oxo-ethoxy]-benzyl}-1,5-dihydro-pyrazolo[3,4-d]pyrimidin-4-one C₂₅H₃₂N₆O₃. MS 465 (M+H)⁺. LC retention time: 1.6 min.

### Example 18

2-[2-(1-Cyclopentyl-4-oxo-4,5-dihydro-1H-pyrazolo[3,4-d]pyrimidin-6-ylmethyl)-phenoxy]-N,N-diethyl-acetamide C₂₃H₂₉N₅O₃. MS 424 (M+H)⁺. LC retention time: 2.4 min.

### Example 19

[2-(3-Isopropyl-7-oxo-6,7-dihydro-1H-pyrazolo[4,3-d]pyrimidin-5-ylmethyl)-phenoxy]-acetic acid

Potassium t-butoxide (2.9 g, 26 mmol) was added to a solution of 5-isopropyl-4-{2-[2-(ethoxy-carbonyl-methoxy)-phenyl]-acetylamino}-1H-pyrazole-3-carboxylic acid amide (950 mg, 2.44 mmol) in dry isopropanol (20 mL). The mixture was heated at 85° C. for 20 h. The reaction was acidified with 1N HCl and extracted with 1:1 EtOAc/THF (3×). The combined extracts were washed with brine, dried (MgSO₄), filtered and concentrated to afford 770 mg of the title compound as a colorless solid.

MS 343 (M+H)⁺; ¹H NMR (400 MHz, CD₃OD) δ 7.22 (m, 2H); 6.95 (m, 2H); 4.77 (s, 2H); 4.08 (s, 2H); 3.4 (m, 1H); 1.4 (d, 6H, J=7.1 Hz). C₁₇H₁₈N₄O₄. LC retention time: 1.7 min.

The following compound, Example 20, was prepared from the appropriate starting material according to the general procedure above for the preparation of Example 19.

### Example 20

[2-(1-Cyclopentyl-4-oxo-4,5-dihydro-1H-pyrazolo[3,4-d]pyrimidin-6-ylmethyl)-phenoxy]-acetic acid C₁₉H₂₀N₄O₄. MS 369 (M+H)⁺. LC retention time: 2.9 min.

### Example 21

3-Isopropyl-5-[2-(5-chloro-2-morpholin-4-yl-ethoxy)-benzyl]-1,6-dihydro-pyrazolo[4,3-d]pyrimidin-7-one

Ozone was bubbled through a solution of 3-isopropyl-5-[2-(5-chloro-2-allyloxy)-benzyl]-1,6-dihydro-pyrazolo[4,3-d]pyrimidin-7-one (134 mg, 0.373 mmol) in CH₂Cl₂ (8 mL) and MeOH (2 mL) at −78° C. until the blue color persisted for 1 min. After purging with nitrogen, dimethyl sulfide (0.4 mL) was added and the reaction allowed to warm to RT. The mixture was concentrated in vacuo, then reconcentrated from CH₂Cl₂ three times. The residue was dissolved in methanol (8 mL) and dioxane (8 mL) and morpholine (65 uL, 0.75 mmol), sodium cyanoborohydride (47 mg, 0.75 mmol), and acetic acid (45 uL) were added. The reaction stirred at RT for 20 h and was then quenched by the addition of 2N HCl. The mixture was made basic by the addition of sat NaHCO₃ solution and then extracted with 1:1 EtOAcTHF (2×). The combined organic layers were washed with brine, dried (MgSO₄), filtered and concentrated in vacuo. Flash chromatography (2→3% MeOH/CH₂Cl₂) afforded 78 mg title compound as a colorless solid.

MS 432 (M+H)⁺; ¹H NMR (400 MHz, CD₃OD) δ 7.22 (m, 2H); 6.96 (d, 1H, J=9.7 Hz); 4.1 (m, 2H); 3.98 (bs, 2H); 3.58 (m, 4H); 2.7 (m, 2H); 2.43 (m, 4H); 1.3 (d, 6H, J=6.6 Hz). C₂₁H₂₆ClN₅O₃. LC retention time: 1.2 min.

The following compounds, Examples 22-25, were prepared from the appropriate olefin and amine according to the general procedure above for the preparation of Example 21.

### Example 22

3-Isopropyl-5-[2-(2-pyrrolidin-1-yl-ethoxy)-benzyl]-1,6-dihydro-pyrazolo[4,3-d]pyrimidin-7-one. C₂₁H₂₅N₅O₃. MS 382 (M+H)⁺. LC retention time: 1.2 min.

### Example 23

3-Isopropyl-5-[2-(2-morpholin-4-yl-ethoxy)-cyclohexylmethyl]-1,6-dihydro-pyrazolo[4,3-d]pyrimidin-7-one hydrochloride. C₂₁H₃₃N₅O₃HCl. MS 404 (M+H)⁺. LC retention time: 1.1 min.

### Example 24

5-[5-Fluoro-2-(2-morpholin-4-yl-ethoxy)y-benzyl]-3-isopropyl-1,6-dihydro-pyrazolo[4,3-d]pyrimidin-7-one hydrochloride. C₂₁H₂₆FN₅O₃.HCl. MS 416 (M+H)⁺. LC retention time: 1.2 min.

### Example 25

3-Cyclopentyl-5-[5-fluoro-2-(2-morpholin-4-yl-ethoxy)-benzyl]-1,6-dihydro-pyrazolo[4,3-d]pyrimidin-7-one hydrochloride. C₂₃H₂₈FN₅O₃.HCl. MS 442 (M+H)⁺. LC retention time: 1.4 min.

### Example 26

3-Isopropyl-5-[2-(2-morpholin-4-yl-ethoxy)-benzyl]-1,6-dihydro-pyrazolo[4,3-d]pyrimidin-7-one hydrochloride.

Potassium t-butoxide (5.9 g, 48 mmol) was added to a solution of 5-isopropyl-4-{2-[2-(2-morpholin-4-yl-ethoxy)-phenyl]-acetylamino}-1H-pyrazole-3-carboxylic acid amide (4.0 g, 9.65 mmol) in dry isopropanol (50 mL). The mixture was heated at 85° C. for 20 h. The reaction was concentrated, diluted with brine and extracted with 1:1 EtOAc/THF (3×). The combined extracts were washed with brine, dried (MgSO₄), filtered and concentrated. Flash chromatography (2→3.5% MeOH/CH₂Cl₂ with 0.2% NH₄OH) afforded the free base. The product was dissolved in ethanol and treated with excess HCl/ether. The mixture was concentrated to afford 4.1 g of the title compound as a colorless solid.

MS 398 (M+H)⁺; ¹H NMR (400 MHz, CD₃OD) δ 7.34 (t, 1H, J=8.3 Hz); 7.22 (d, 1H, J=7.5 Hz); 7.07 (d, 1H, J=8.3 Hz); 7.01 (d, 1H, J=7.5 Hz); 4.41 (m, 2H); 4.2 (s, 2H); 3.9 (bm, 4H); 3.61 (m, 2H); 3.4 (m, 1H); 3.29 (m, 4H); 1.35 (d, 6H, J=7.1 Hz). C₂₁H₂₇N₅O₃.HCl. LC retention time: 1.2 min.

The following compounds, Examples 27-31, were prepared from the appropriate amide according to the general procedure above for the preparation of Example 26.

### Example 27

9-(1,2-Dimethyl-propyl)-2-[2-(2-morpholin-4-yl-ethoxy)-benzyl]-1,9-dihydro-purin-6-one. C₂₃H₃₁N₅O₃. MS 426 (M+H)⁺. LC retention time: 1.4 min.

### Example 28

2-[2-(2-Morpholin-4-yl-ethoxy)-benzyl]-9-(tetrahydro-furan-3-yl)-1,9-dihydro-purin-6-one. C₂₂H₂₇N₅O₄. MS 426 (M+H)⁺. LC retention time: 0.7 min.

### Example 29

5-[2-(2-Diethylamino-ethoxy)-benzyl]-3-isopropyl-1,6-dihydro-pyrazolo[4,3-d]pyrimidin-7-one. C₂₁H₂₉N₅O₂. MS 384 (M+H)⁺. LC retention time: 1.1 min.

### Example 30

3-Cyclopentyl-5-[2-(2-morpholin-4-yl-ethoxy)-benzyl]-1,6-dihydro-pyrazolo[4,3-d]pyrimidin-7-one hydrochloride. C₂₃H₂₉N₅O₃.HCl. MS 424 (M+H)⁺. LC retention time: 1.4 min.

### Example 31

3-Cyclobutyl-5-[2-(2-morpholin-4-yl-ethoxy)-benzyl]-1,6-dihydro-pyrazolo[4,3-d]pyrimidin-7-one hydrochloride. C₂₂H₂₇N₅O₃. MS 410 (M+H)⁺. LC Retention time: 2.1 min.

### Example 32

9-(1(R),2-Dimethyl propyl)-[2-(2-morpholin-4-yl-ethoxy)-benzyl]-1,9-dihydro-purin-6-one hydrochloride

Potassium t-butoxide (143 mg, 1.27 mmol) was added to a solution of 5-amino-1-(1(R),2-dimethyl propyl)-1H-imidazole-4-carboxylic acid amide (50 mg, 0.25 mmol) and (2-morpholin-4-yl-ethoxy) phenyl acetic acid methyl ester (285 mg, 1.02 mmol) in anhydrous 1-butanol (2 mL). The mixture was heated in a microwave apparatus for 30 min at 180° C. The mixture was concentrated and purified by flash chromatography (1→4% MeOH/CH₂Cl₂). The purified product was dissolved in methanol/ether and treated with excess HCl/ether. The resulting solid was filtered and dried to afford 73 mg of the title compound as a colorless solid.

MS 426 (M+H)⁺; ¹H NMR (400 MHz, CD₃OD) δ 9.25 (s, 1H); 7.32 (t, 1H, J=8.3 Hz); 7.22 (d, 1H, J=7.5 Hz); 7.03 (d, 1H, J=8.3 Hz); 7.0 (t, 1H, J=7.5 Hz), 4.4 (m, 3H); 4.18 (s, 2H); 4.0 (m, 2H); 3.81 (m, 2H); 3.61 (m, 2H); 3.59 (m, 2H); 2.12 (m, 2H); 1.55 (d, 3H, J=7.1 Hz); 0.91 (d, 3H, J=6.6 Hz); 0.72 (d, 3H, J=6.6 Hz). C₂₃H₃lN₅O₃.HCl. LC retention time: 1.4 min.

The following compounds, Examples 33-44, were prepared from the appropriate amine and ester according to the general procedure above for the preparation of Example 32.

### Example 33

9-(2-Methyl-butyl)-2-[2-(2-morpholin-4-yl-ethoxy)-benzyl]-1,9-dihydro-purin-6-one. C₂₃H₃₁N₅O₃. MS 426 (M+H)⁺. LC retention time: 2.4 min.

### Example 34

9-Cyclopentyl-2-[2-(2-morpholin-4-yl-ethoxy)-benzyl]-1,9-dihydro-purin-6-one. C₂₃H₂₉N₅O₃. MS 424 (M+H)⁺. LC retention time: 2.3 min.

### Example 35

5-[2-(2-Morpholin-4-yl-ethoxy)-benzyl]-3-pyridin-3-yl-1,6-dihydro-pyrazolo[4,3-d]pyrimidin-7-one. C₂₃H₂₄N₆O₃. MS 433 (M+H)⁺. LC retention time: 2.0 min.

### Example 36

9-(1,2-Dimethyl-propyl)-2-[2-(2-morpholin-4-yl-ethoxy)-benzyl]-1,9-dihydro-purin-6-one hydrochloride. C₂₃H₃₁N₅O₃.HCl. MS 426 (M+H)⁺. LC retention time: 1.4 min.

### Example 37

9-Isopropyl-2-[2-(2-morpholin-4-yl-ethoxy)-benzyl]-1,9-dihydro-purin-6-one hydrochloride. C₂₁H₂₇N₅O₃.HCl. MS 398 (M+H)⁺. LC retention time: 1.0 min.

### Example 38

2-[2-(2-Morpholin-4-yl-ethoxy)-benzyl]-9-(tetrahydro-furan-2-ylmethyl)-1,9-dihydro-purin-6-one hydrochloride. C₂₃H₂₉N₅O₄.HCl. MS 440 (M+H)⁺. LC retention time: 0.9 min.

### Example 39

9-(1-Isopropyl-2-methyl-propyl)-2-[2-(2-morpholin-4-yl-ethoxy)-benzyl]-1,9-dihydro-purin-6-one hydrochloride. C₂₅H₃₅N₅O₃.HCl. MS 454 (M+H)⁺. LC retention time: 1.7 min.

### Example 40

9-(1-Ethyl-propyl)-2-[2-(2-morpholin-4-yl-ethoxy)-benzyl]-1,9-dihydro-purin-6-one hydrochloride. C₂₃H₃₁N₅O₃.HCl. MS 426 (M+H)⁺. LC retention time: 1.4 min.

### Example 41

9-Cyclopentyl-8-methyl-2-[2-(2-morpholin-4-yl-ethoxy)-benzyl]-1,9-dihydro-purin-6-one hydrochloride. C₂₄H₃₁N₅O₃. MS 438 (M+H)⁺. LC Retention time: 1.4 min.

### Example 42

3-Cyclopentyl-5-[2-(2-morpholin-4-yl-ethoxy)-benzyl]-3,6-dihydro-[1,2,3]triazolo[4,5-d]pyrimidin-7-one hydrochloride. C₂₂H₂₈N₆O₃. MS 425 (M+H)⁺. LC Retention time: 1.3 min.

### Example 43

1-Cyclopentyl-6-[2-(2-morpholin-4-yl-ethoxy)-benzyl]-1,5-dihydro-pyrazolo[3,4-d]pyrimidin-4-one hydrochloride. C₂₃H₂₉N₅O₃. MS 424 (M+H)⁺. LC Retention time: 1.3 min.

### Example 44

9-Cyclopentyl-2-[2-(3-morpholin-4-yl-propoxy)-benzyl]-1,9-dihydro-purin-6-one hydrochloride. C₂₄H₃₁N₅O₃. MS 438 (M+H)⁺. LC Retention time: 2.4 min.

### Example 45

N-[(1R,2S)2-(3-Isopropyl-7-oxo-6,7-dihydro-1H-pyrazolo[4,3-d]pyrimidin-5-ylmethyl-cyclohex-1-yl]-2-pyrrolidin-1-yl-acetamide hydrochloride.

Pyrrolidine (70 μL, 0.83 mmol) was added to a mixture of N-[(1R,2S)2-(3-isopropyl-7-oxo-6,7-dihydro-1H-pyrazolo[4,3-d]pyrimidin-5-ylmethyl)-cyclohex-1-yl]-2-chloro-acetamide (76 mg, 0.208 mmol) in anhydrous THF (3 mL) at RT. The mixture was heated at 65° C. for 2.5 h, then allowed to cool to RT and stir for 16 h. The reaction was concentrated and purified by flash chromatography (2→3→4% MeOH/CH₂Cl₂ with 0.2% NH₄OH). The product was dissolved in methanol/ether and treated with excess HCl/ether. The resulting solid was filtered and dried to afford 20 mg of the title compound as a colorless solid.

MS 401 (M+H)⁺; ¹H NMR (400 MHz, CD₃OD) δ 4.07 (s, 2H); 3.7 (m, 3H); 3.45 (m, 1H); 3.14 (m, 3H); 2.59 (dd, 1H, J=14.2, 9.2 Hz); 2.2-1.7 (m, 9H); 1.4 (d, 6H, J=7.1 Hz); 1.4-1.2 (m, 4H). C₂₁H₃₂N₆O₂.HCl. LC retention time: 1.0 min.

The following compounds, Examples 46-50, were prepared from the appropriate amine according to the general procedure above for the preparation of Example 45.

### Example 46

N-[(1R,2S)2-(3-Isopropyl-7-oxo-6,7-dihydro-1H-pyrazolo[4,3-d]pyrimidin-5-ylmethyl)-cyclohex-1-yl]-2-morpholin-4-yl-acetamide hydrochloride. C₂₁H₃₂N₆O₃. MS 417 (M+H)⁺. LC Retention time: 1.1 min.

### Example 47

2-Diethylamino-N-[(1R,2S)2-(3-isopropyl-7-oxo-6,7-dihydro-1H-pyrazolo[4,3-d]pyrimidin-5-ylmethyl)-cyclohex-1-yl]-acetamide hydrochloride. C₂₁H₃₄N₆O₂. MS 403 (M+H)⁺. LC Retention time: 1.13 min.

### Example 48

1-{[(1R,2S)2-(3-Isopropyl-7-oxo-6,7-dihydro-1H-pyrazolo[4,3-d]pyrimidin-5-ylmethyl)-cyclohex-1-ylcarbamoyl]-methyl}-pyrrolidine-2(S)-carboxylic acid methyl ester hydrochloride. C₂₃H34N₆O₄. MS 459 (M+H)⁺. LC Retention time: 1.4 min.

### Example 49

2-Cyclobutylamino-N-[(1R,2S)2-(3-isopropyl-7-oxo-6,7-dihydro-1H-pyrazolo[4,3-d]pyrimidin-5-ylmethyl)-cyclohex-1-yl]-acetamide hydrochloride. C₂₁H₃₂N₆O₂. MS 401 (M+H)⁺. LC Retention time: 1.2 min.

### Example 50

2-Cyclorropylamino-N-[(1R,2S)2-(3-isopropyl-7-oxo-6,7-dihydro-1H-pyrazolo[4,3-d]pyrimidin-5-ylmethyl)-cyclohex-1-yl]-acetamide hydrochloride. C₂₀H₃₀N₆O₂. MS 387 (M+H)⁺. LC Retention time: 1.1 min.

The following enumerated Preparations describe the preparation of certain intermediates used in the preceding Examples.

### Preparation A1

N-[(1R,2S)2-(3-isopropyl-7-oxo-6,7-dihydro-1H-pyrazolo[4,3-d]pyrimidin-5-ylmethyl)cyclohex-1-yl]-2-chloro-acetamide.

Chloroacetyl chloride (95 μL, 1.2 mmol) was added to a suspension of 5-(2-amino-cyclohex-1-ylmethyl)-3-isopropyl-1,6-dihydro-pyrazolo[4,3-d]pyrimidin-7-one (200 mg, 0.614 mmol) and pyridine (1.2 mL, 14.8 mmol) in dry CH₂Cl₂ (8 mL) and dioxane (3 mL) at RT. After 24 h, 1N HCl was added to the mixture and it was extracted with EtOAc (2×). The combined extracts were washed with 1N HCl and brine, dried (MgSO₄), filtered and concentrated to afford 76 mg of the title compound as a tan solid.

MS 366 (M+H)⁺; ¹H NMR (400 MHz, d6 acetone) δ 8.6 (bs, 1H); 7.5 (bd, 1H, J=4.5 Hz); 7.35 (m,1H); 4.02 (s, 2H); 3.42 (m, 1H); 3.3 (m, 1H); 2.95 (dd, 1H, J=14.6, 5.0 Hz); 2.5 (dd, 1H, J=14.6, 7.5 Hz); 1.9 (m, 2H); 1.65 (m, 2H); 1.4 (d, 6H, J=7.0 Hz); 1.4-1.05 (m, 5H).

### Preparation B1

5-((1S,2R)2-Amino-cyclohex-1-ylmethyl)-3-isopropyl-1,6-dihydro-pyrazolo[4,3-d]pyrimidin-7-one.

10% Pd/C (70 mg) was added to a solution of 5-((1S,2R)2-azido-cyclohex-1-ylmethyl)-3-isopropyl-1,6-dihydro-pyrazolo[4,3-d]pyrimidin-7-one (408 mg, 12.9 mmol) in absolute ethanol (30 mL) containing 1N HCl (2 mL). The mixture was placed under 45 psi of hydrogen for 2 h, and then the reaction was purged with nitrogen and filtered. The filtrate was concentrated in vacuo, triturated with ether, filtered and dried to afford 322 mg of the title compound as a colorless solid.

MS 290 (M+H)⁺; ¹H NMR (400 MHz, CD₃OD) δ 3.39 (m, 1H); 3.12 (m, 1H); 2.95 (m, 1H); 2.69 (m, 1H); 2.01 (m, 2H); 1.85 (m, 2H); 1.73 (m, 1H); 1.5-1.2 (m, 4H); 1.4 (d, 6H, J=7.0 Hz).

### Preparation C1

5-((1S,2R)2-Azido-cyclohex-1-ylmethyl)-3-isopropyl-1,6-dihydro-pyrazolo[4,3-d]pyrimidin-7-one

Potassium t-butoxide (695 mg, 5.68 mmol) was added to a solution of 5-isopropyl-4-[2-((1S,2R)2-azido-cyclohex-1-yl)-acetylamino]-1H-pyrazole-3-carboxylic acid amide (379 mg, 1.14 mmol) in dry isopropanol (10 mL). The mixture was heated at 85° C. for 20 h. The reaction was concentrated in vacuo and the residue was acidified with 1N HCl and extracted with EtOAc (3×). The combined extracts were washed with brine, dried (MgSO₄), filtered and concentrated to afford 437 mg of the title compound as a colorless solid.

MS 316 (M+H)⁺; ¹H NMR (400 MHz, CD₃OD) δ 3.4 (bm, 1H); 3.15 (dt, 1H, J=10.4, 4.2 Hz); 2.97 (dd, 1H, J=14.2, 5.3 Hz); 2.48 (dd, 1H, J=14.2, 8.3 Hz); 2.15-1.5 (m, 9H); 1.4 (d, 6H, J=7.0 Hz).

### Preparation D1

5-Isopropyl-4-{2-[2-(2-morpholin-4-yl-ethoxy)-phenyl]-acetylamino}-1H-pyrazole-3-carboxylic acid amide.

1-Propanephosphonic acid cyclic anhydride (50% solution in ethyl acetate, 7.3 mL, 0.012 mol) was added to a solution of 4-amino-5-isopropyl-1H-pyrazol-3-carboxylic acid amide (1.7 g, 10 mmol), 2-(morpholin-4-yl-ethoxy)-phenyl acetic acid (3.5 g, 13 mmol), and triethyl amine (2.85 mL, 20 mmol) in anhydrous DMF (30 mL) at RT. After 20 h, the mixture was concentrated in vacuo, and the residue purified by flash chromatography (2→6% MeOH/CH₂Cl₂ with 0.2% NH₄OH) afforded 4.0 g of the title compound as a colorless solid.

MS 416 (M+H)⁺; ¹H NMR (400 MHz, CD₃OD) δ 7.25 (m, 2H); 6.99 (d, 1H, J=6.9 Hz); 6.95 (t, 1H, J=7.5 Hz); 4.19 (m, 2H); 3.7 (m, 2H); 3.59 (m, 2H); 3.01 (m, 1H); 2.8 (m, 2H); 2.56 (m, 2H); 1.2 (d, 6H, J=6.6 Hz).

The following compounds, Preparations D2-D6, were prepared from the appropriate amine and acid according to the general procedure described above concerning the preparation of D1.

### Preparation D2

5-Cyclopentyl-4-{2-[2-(2-morpholin-4-yl-ethoxy)-phenyl]-acetylamino}-1H-pyrazole-3-carboxylic acid amide.

### Preparation D3

5-Isopropyl-4-{2-[2-(2-diethylamino-ethoxy)-phenyl]-acetylamino}-1H-pyrazole-3-carboxylic acid amide.

### Preparation D4

1-(12-Dimethyl-propyl)-5-{2-[2-(2-morpholin-4-yl-ethoxy)-phenyl]-acetylamino}-1H-imidazole-4-carboxylic acid amide

### Preparation D5

5-{2-[2-(2-Morpholin-4-yl-ethoxy)-phenyl]-acetylamino}-1-(tetrahydro-furan-3-yl)-1H-imidazole-4-carboxylic acid amide

### Preparation D6

4-[2-(2-Amino-cyclohexyl)-acetylamino]-5-isopropyl-2H-pyrazole-3-carboxylic acid amide

### Preparation E1

4-Amino-5-isopropyl-1H-pyrazol-3-carboxylic acid amide

5-Isopropyl-4-nitro-1H-pyrazol-3-carboxylic acid amide (3 g, 15.1 mmol) and 10% palladium on carbon (500 mg) in ethanol (30 mL) were stirred under hydrogen (50 psi) at RT for 18 h. The reaction mixture was filtered and the solid was washed with methanol (50 mL), dichloromethane (50 mL), ethanol (50 mL) and ethyl acetate (50 mL). The filtrate was concentrated under reduced pressure and the residue was purified by flash column chromatography on silica gel eluting with dichloromethane: methanol (9:1, by volume) to give the title product (2.6 g) as an off-white solid; ¹H NMR (400 MHz, DMSO-D6): δ 12.20-12.30 (bs, 1H), 7.02-7.14 (bs, 1H), 6.85-6.95 (bs, 1H), 4.30-4.46 (bs, 2H), 2.90-3.00 (m, 1H), 1.15-1.21 (d, 6H) ppm; LRMS (electrospray): m/z [M−H]⁺ 167, [2M−H]⁺ 335; Anal. Found C, 49.86; H, 7.21; N, 33.07. C₇H₁₂N₄O requires C, 49.99; H, 7.19; N, 33.31%.

The following compounds, Preparations E2-E3, were prepared from the appropriate nitro pyrazole according to the general procedure above for the preparation of E1.

### Preparation E2

4-Amino-5-cyclopentyl-1H-pyrazol-3-carboxylic acid amide

### Preparation E3

4-Amino-5-cyclobutyl-1H-pyrazol-3-carboxylic acid amide

### Preparation F1

5-Isopropyl-4-nitro-1H-pyrazol-3-carboxylic acid amide

Oxalyl chloride (6.8 mL, 77.6 mmol) was added dropwise to a suspension of 5-isopropyl-4-nitro-1H-pyrazol-3-carboxylic acid (5.15 g, 25.9 mmol) in dichloromethane (80 mL) containing dimethylformamide (0.1 mL) under nitrogen at 0° C. The reaction was stirred at 0° C. for 1 h, allowed to warm to RT and stirred for a further 2 h. The solvent was removed under reduced pressure, the residue was dissolved in toluene (100 mL) and ammonia gas was bubbled into the solution for 2 h. The reaction was stirred under nitrogen at room temperature for 18 h, concentrated under reduced pressure and the residue was dissolved in hot methanol (300 mL). The resultant precipitate was filtered and the filtrate was concentrated under reduced pressure. The residue was azeotroped with water (300 mL), concentrated to approximately 80 mL under reduced pressure and the precipitate was isolated by filtration. This was washed with water and dried under vacuum to give the title product (3.1 g) as an orange solid; ¹H NMR (400MHz, DMSO-D6): δ 7.94-7.99 (bs, 1H); 7.68-7.72 (bs, 1H); 3.45-3.55 (m, 1H), 1.24-1.30 (d, 6H) ppm; LRMS (electrospray): m/z [M+Na]⁺ 221, [M−H]⁺ 197.

The following compounds, Preparations F2-F3, were prepared from the appropriate nitro pyrazole according to the general procedure above for the preparation of F1.

### Preparation F2

5-Cyclopentyl-4-nitro-1H-pyrazol-3-carboxylic acid amide

### Preparation F3

5-Cyclobutyl-4-nitro-1H-pyrazol-3-carboxylic acid amide

### Preparation G1

5-Isopropyl-4-nitro-1H-pyrazol-3-carboxylic acid

5-isopropyl-1H-pyrazol-3-carboxylic acid (5 g, 32.5 mmol) was added portionwise to concentrated sulfuric acid (25 mL) at RT with stirring. The reaction mixture was then heated to 60° C. and concentrated nitric acid (70%, 6 mL, 90 mmol) was added dropwise, keeping the temperature at 60° C. The reaction was then stirred at 60° C. for 3 h, cooled to RT and poured onto 50 mL of ice with stirring. After 15 min the white precipitate was isolated by filtration, washed with water and dried under reduced pressure to give the title product (5.2 g) as a white solid; ¹H NMR (400 MHz, DMSO-D6): 6 13.86-13.93 (bs, 1H), 13.50-13.80 (bs, 1H), 3.39-3.52 (m, 1H), 1.18-1.30 (d, 6H) ppm; LRMS (electrospray): m/z [M−H]⁺ 198.

The following compounds, Preparations G2-G3, were prepared from the appropriate pyrazole according to the general procedure above for the preparation of G1.

### Preparation G2

5-Cyclopentyl-4-nitro-1H-pyrazol-3-carboxylic acid

### Preparation G3

5-Cyclobutyl-4-nitro-1H-pyrazol-3-carboxylic acid

### Preparation H1

5-Isopropyl-1H-pyrazol-3-carboxylic acid

5-isopropyl-1H-pyrazol-3-carboxylic acid ethyl ester (18.9 g, 104 mmol) and 1M NaOH solution (260 mL, 259 mmol) were dissolved in 1,4-dioxane (300 mL), the reaction was heated to 50° C. under nitrogen and stirred for 3 h. The reaction mixture was cooled, adjusted to pH 2 using concentrated hydrochloric acid and the solvent was removed under reduced pressure. The residual solid was azeotroped with toluene (2×30 mL), dissolved in ethyl acetate (500 mL) and washed with water. The aqueous phase was removed, extracted with ethyl acetate (2×200 mL) and the combined organic extracts were dried over MgSO₄. The solvent was removed under reduced pressure and the residue was azeotroped with dichloromethane (2×50 mL) to give the title product (14.7 g) as a white solid; ¹H NMR (400 MHz, DMSO-D6): δ 12.50-13.30 (bs, 2H), 6.42 (s, 1H), 2.84-2.94 (quin, 1H), 1.15-1.19 (d, 6H) ppm; LRMS (electrospray): m/z [M−H]⁺ 153.

The following compounds, Preparations H2-H3, were prepared from the appropriate ester according to the general procedure above for the preparation of H1.

### Preparation H2

5-Cyclopentyl-1H-pyrazol-3-carboxylic acid

### Preparation H3

5-Cyclobutyl-1H-pyrazol-3-carboxylic acid

### Preparation I1

5-Isopropyl-1H-pyrazol-3-carboxylic acid ethyl ester

Hydrazine hydrate (6.6 mL, 134 mmol) was added to a solution of 5-methyl-2,4-dioxo-hexanoic acid ethyl ester (23.8 g, 188 mmol) in ethanol (100 mL) at RT under nitrogen. The reaction was allowed to proceed at RT for 18 h, and the solvent was removed under reduced pressure. The residue was partitioned between dichloromethane (300 mL) and water (300 mL) and the aqueous phase was removed. The organic phase was washed with water (2×200 mL), dried over MgSO₄ and concentrated under reduced pressure. The residue was purified by flash column chromatography on silica gel eluting with a solvent gradient of pentane:ethyl acetate (4:1 changing to 2:1, by volume) to give the title product (18.9 g) as a white solid; ¹H NMR (400 MHz, CDCl₃): δ 10.80-10.95 (bs, 1H), 6.61 (s, 1H), 4.33-4.40 (quart, 2H), 2.98-3.08 (quin, 1H), 1.35-1.41 (t, 3H), 1.24-1.32 (d, 6H) ppm; LRMS (electrospray): m/z [M−H]⁺ 181.

The following compounds, Preparations 12-13, were prepared from the appropriate diketone according to the general procedure above for the preparation of I1.

### Preparation I2

5-Cyclopentyl-1H-pyrazol-3-carboxylic acid ethyl ester

### Preparation I3

5-Cyclobutyl-1H-pyrazol-3-carboxylic acid ethyl ester

### Preparation J1

5-Methyl-2,4-dioxo-hexanoic acid ethyl ester

Sodium pellets (3.39 g, 148 mmol) were dissolved in ethanol (100 mL) under nitrogen at RT and a solution of diethyloxalate (20 ml, 147 mmol) in 3-methyl-2-butanone (18.9 ml, 177 mmol) was added dropwise at RT over 30 min. The reaction was diluted with ethanol (100 mL), heated to 60° C. and stirred at this temperature for 2 h. After cooling to room temperature the reaction was poured onto ice-cold 2N HCl (200 mL) and extracted with diethylether (300 mL) and ethyl acetate (300 mL). The combined organic extracts were dried over MgSO₄, concentrated under reduced pressure and the residue was purified by flash column chromatography on silica gel eluting with a solvent gradient of pentane:ethyl acetate (99:1 changing to 95:5, by volume) to give the title product (23.8 g) as a yellow oil; ¹H NMR (400MHz, CDCl₃): δ 14.40-14.80 (bs, 1H), 6.40 (s, 1H), 4.30-4.39 (quart, 2H), 2.60-2.71 (quin, 1H), 1.35-1.40 (t, 3H), 1.15-1.20 (d, 6H) ppm; LRMS (electrospray): m/z [M−H]⁺ 185.

The following compounds, Preparations J2-J3, were prepared from the appropriate ketone according to the general procedure above for the preparation of J1.

### Preparation J2

4-Cyclopentyl-2,4-dioxo-butanoic acid ethyl ester

### Preparation J3

4-Cyclobutyl-2,4-dioxo-butanoic acid ethyl ester

### Preparation K1

5-Amino-1-(1,2-dimethyl propyl)-1H-imidazole-4-carboxylic acid amide

A mixture of 2-cyano-2-amino acetamide (200 mg, 2 mmol) and triethyl orthoformate (0.37 mL, 2.42 mmol) in dry acetonitrile (5 mL) was heated at 80° C. for 3 h. 1,2-dimethyl propyl amine (211 mg, 2.4 mmol) was added and the reaction was allowed to cool to RT and stirred overnight. The mixture was concentrated and purified by flash chromatography (1→3% MeOH/CH₂Cl₂) to afford 221 mg of the title compound as a tan solid.

MS 197 (M+H)⁺; ¹H NMR (400 MHz, d6 DMSO) δ 7.18 (s, 1H); 6.68 (bs, 1H); 6.58 (bs, 1H); 5.71 (bs, 2H); 3.8 (m, 1H); 1.93 (m, 1H); 1.3 (d, 3H, J=7.1 Hz); 0.87 (d, 3H, J=7.1 Hz); 0.7 (d, 3H, J=6.6 Hz).

The following compounds, Preparation K2-K11, were prepared from the appropriate amine and orthoformate according to the general procedure above for the preparation of K1.

### Preparation K2

5-Amino-1-(1(S),2-dimethyl propyl)-1H-imidazole-4-carboxylic acid amide

### Preparation K3

5-Amino-1-(1(R),2-dimethyl propyl)-1H-imidazole-4-carboxylic acid amide

### Preparation K4

5-Amino-1-(1-methyl butyl)-1H-imidazole-4-carboxylic acid amide

### Preparation K5

5-Amino-1-(cyclopentyl)-1H-imidazole-4-carboxylic acid amide

### Preparation K6

5-Amino-1-(isopropyl)-1H-imidazole-4-carboxylic acid amide

### Preparation K7

5-Amino-1-(1-isopropyl-2-methyl propyl)-1H-imidazole-4-carboxylic acid amide

### Preparation K8

5-Amino-1-(2-ethyl propyl)-1H-imidazole-4-carboxylic acid amide

### Preparation K9

5-Amino-1-(tetrahydrofuran-3-yl)-1H-imidazole-4-carboxylic acid amide

### Preparation K10

5-Amino-1-(tetrahydrofuran-3-yl-methyl)-1H-imidazole-4-carboxylic acid amide

### Preparation K11

5-Amino-1-cyclopentyl-2-methyl-1H-imidazole-4-carboxylic acid amide

### Preparation L1

5-Amino-1-cyclopentyl-1H-pyrazole-4-carboxylic acid amide

Concentrated H₂SO₄ (14.7 mL) was added to 5-amino-1-cyclopentyl-1H-pyrazole-4-carbonitrile (5.18 g, 29.4 mmol) at RT. The reaction was stirred at RT for 44 h. The reaction was poured onto ice and the pH was adjusted to 11 by addition of NH₄OH. The solid was filtered to afford 3.74 g of the title compound as a brown solid. MS 195 (M+H)⁺. ¹HNMR (400 MHz, CD₃OD) δ 7.69 (s, 1H); 4.54 (m, 1H); 2.08-1.69 (m, 8H).

### Preparation M1

5-Amino-1-cyclopentyl-1H-pyrazole-4-carbonitrile

To a solution of sodium ethoxide (0.34 g of Na in 30 mL of absolute ethanol) was added cyclopentyl-hydrazine hydrochloride (2.0 g, 14.6 mmol) at RT. The reaction was heated to 75° C. for 2 h. The reaction was allowed to cool to RT and stirred for 12 h. The solvent was removed in vacuo to give an oily residue which was dissolved in 50 mL of water and extracted with EtOAc. The combined organic layers were washed with brine, dried (MgSO₄), and concentrated to afford 1.69 g of the title compound as an orange solid. MS 177 (M+H)⁺. ¹HNMR (400 MHz, CD₃OD) δ 7.05 (s, 1H); 4.51 (m, 1H); 2.05-1.57 (m, 8H).

### Preparation N1

Cyclopentyl-hydrazine hydrochloride

Borane-tetrahydrofuran complex (105.4 mL of a 1M solution in THF, 105.4 mmol) was added to N′-cyclopentylidene-hydrazinecarboxylic acid tert-butyl ester (20.89 g, 105.4 mmol) at RT and stirred for 15 minutes while gas evolved. 6N HCl (52.7 mL) was added slowly and reaction was stirred at RT for 10 minutes. The reaction was heated to 80° C. for 15 minutes, then cooled to RT and concentrated to dryness. The resulting solid was washed with THF (250 mL) and a white solid was removed by filtration. The filtrate was concentrated to afford 12.7 g of the title compound as a white solid. MS 101 (M+H)⁺. ¹H NMR (400 MHz, CD₃OD) δ 3.57 (m, 1H); 2.02 (m, 2H); 1.77-1.62 (m, 6H).

### Preparation O1

N′-Cyclopentylidene-hydrazinecarboxylic acid tert-butyl ester

t-butyl carbazate (14.9 g, 113 mmol) in hexanes (100 mL) was heated to 75° C. Cyclopentanone (10.0 mL, 113 mmol) was added and the reaction was maintained at 75° C. for 1 hour. The reaction was allowed to cool to RT. The crystallized product was collected by filtration to yield 20.89 g of the title compound as a white solid. MS 199 (M+H)⁺. ¹HNMR (400 MHz, CDC1₃) δ 2.57 (m, 2H); 2.27 (m, 2H); 1.88 (m, 2H); 1.79 (m, 2H); 1.52 (s, 9H).

### Preparation P1

5-Amino-1-cyclopentyl-1H-[1,2,3]triazole-4-carboxylic acid amide Sodium ethoxide (prepared from 920 mg of sodium in 40 mL ethanol, 40 mmol) was heated to 70° C. and a solution of azido cyclopentane (2.2 g, 20 mmol) and cyanoacetamide (1.68 g, 20 mmol) in ethanol (5 mL) was added. After 2 h the reaction was cooled and concentrated to remove most of the ethanol. The residue was diluted with pH 7 buffer and extracted with EtOAc (3×). The combined organic layers were washed with brine, dried (Na₂SO₄) filtered, and concentrated to afford 500 mg of the title compound as a tan solid. MS 196 (M+H)⁺; ¹H NMR (400 MHz, CD₃OD) δ 4.6 (m, 1H); 2.1 (m, 4H); 1.95 (m, 2H); 1.7 (m, 2H).

### Preparation Q1

(2-Allyloxy-5-chloro-phenyl)acetic acid

A solution of KOH (2.3 g, 0.04 mol) in ethanol (20 mL) and water (2 mL) was added to (2-allyloxy-5-chloro-phenyl) acetonitrile (1.6 g, 8 mmol) at RT. The mixture was heated at 93° C. for 3.5 h, then concentrated in vacuo. The residue was dissolved in water (5 mL) and treated with concentrated HCl (4 mL) added dropwise at 0° C. The mixture was stirred for 15 min, then diluted with water and extracted with EtOAc (2×). The organic layers were combined and washed with brine, dried (MgSO₄), filtered and concentrated to afford 1.7 g product as a colorless solid.

MS 225 (M−H)⁻; ¹H NMR (400 MHz, d6 acetone) δ 7.28 (d, 1H, J=3 Hz); 7.22 (dd, 1H, J=8.6, 3.0 Hz); 6.98 (d, 1H, J=8.6 Hz); 6.01 (m, 1H); 5.42 (d, 1H, J=17.5 Hz); 5.2 (d, 1H, J=11.4 Hz); 4.6 (m, 2H); 3.6 (s, 2H).

The following compound, Preparation Q2, was prepared from the appropriate nitrile according to the general procedure above for the preparation of Q1.

### Preparation Q2

(2-Allyloxy-5-fluoro-phenyl)acetic acid

### Preparation R1

(2-Allyloxy-5-chloro-phenyl)acetonitrile

Thionyl chloride (3.6 mL, 50 mmol) was added over eight minutes to a solution of 2-allyloxy-5-chloro-benzyl alcohol (5.9 g, 30 mmol) in dry THF (50 mL) containing 0.2 mL of DMF at 0° C. After 2.5 h, the reaction was quenched by the careful addition of water. The mixture was extracted with EtOAc (3×) and the combined extracts were dried (MgSO₄), filtered and concentrated to afford 7.1 g of the corresponding benzyl chloride. This product was dissolved in dry DMF (80 mL) and sodium cyanide (2 g, 40 mmol) was added. The mixture was heated at 80° C. for 2 h, cooled, diluted with water and extracted with EtOAc (3×). The combined extracts were washed with water (3×), and brine, dried (MgSO₄), filtered and concentrated in vacuo. The product was purified by flash chromatography (5→30% EtOAc/hexanes) to afford 2.0 g of the title compound as a colorless oil.

¹H NMR (400 MHz, CDCl₃) δ 7.38 (s, 1H); 7.22 (m, 1H); 6.8 (d, 1H, J=9.1 Hz); 6.01 (m, 1H); 5.4 (d, 1H, J=17.5 Hz); 5.3 (d, 1H, J=10.8 Hz); 4.59 (m, 2H); 3.65 (s, 2H).

The following compound, Preparation R², was prepared from the appropriate benzyl alcohol according to the general procedure above for the preparation of R1.

### Preparation R2

(2-Allyloxy-5-fluoro-phenyl)acetonitile

### Preparation S1

2-Allyloxy-5-chloro-benzyl alcohol

Lithium aluminum hydride (1.17 g, 0.031 mol) was added in portions to a solution of 2-allyloxy-5-chloro-benzoic acid allyl ester (7.4 g, 0.029 mol) in dry THF (100 mL) at 0° C. After 1.5 h, the reaction was quenched by the sequential addition of water (1.2 mL), 15% NaOH (1.2 mL), and water (3.6 mL). The mixture was dried (MgSO₄), filtered, and concentrated to afford 5.9 g of the title compound as a colorless oil.

MS 197 (M−H)⁻; ¹H NMR (400 MHz, d6 acetone) δ 7.42 (d, 1H, J=3 Hz); 7.19 (dd, J=8.5, 3.0 Hz); 6.95 (d, 1H, J=8.5 Hz); 6.03 (m, 1H); 5.4 (d, 1H, J=17.5 Hz); 5.22 (d, 1H, J=11.4 Hz); 4.63 (m, 2H); 4.6 (m, 2H).

The following compound, Preparation S2, was prepared from the appropriate ester according to the general procedure above for the preparation of S1.

### Preparation S2

2-Allyloxy-5-fluoro-benzyl alcohol

### Preparation T1

2-Allyloxy-5-chloro-benzoic acid allyl ester

Cesium carbonate (20.8 g, 63.7 mmol) was added to a mixture of 5-chloro-2-hydroxy-benzoic acid (5 g, 29 mmol) and allyl bromide (5.5 mL, 64 mmol) in dry DMF (60 mL) at RT. After 20 h, the mixture was diluted with water and extracted with ether (3×). The combined organic extracts were washed with water and brine, dried (MgSO₄), filtered and concentrated in vacuo to provide 7.4 g of the title compound as an oil.

MS 253 (M+H)⁺; ¹H NMR (400 MHz, d6 acetone) δ 7.7 (d, 1H, J=2.8 Hz); 7.52 (dd, 1H, J=8.5, 2.8 Hz); 7.18 (d, 1H, J=8.5 Hz); 6.02 (m, 1H); 5.5 (dd, 1H, J=17.5, 1.6 Hz); 5.41 (d, 1H, J=17.5 Hz); 5.22 (d, 2H, J=10.3 Hz); 4.8 (d, 2H, J=5.4 Hz); 4.63 (m, 2H).

The following compound, Preparation T2, was prepared from the appropriate acid according to the general procedure above for the preparation of T1.

### Preparation T2

2-Allyloxy-5-fluoro-benzoic acid allyl ester

### Preparation U1

[2-(3-Morpholin-4-yl-propoxy)-phenyl]-acetic acid methyl ester

Morpholine (90 μL, 1.05 mmol) was added to a mixture of [2-(3-bromo-propoxy)-phenyl]-acetic acid methyl ester (300 mg, 1.05 mmol) and sodium bicarbonate (132 mg, 1.58 mmol) in dry acetonitrile (3 mL). The mixture was heated at 85° C. overnight then concentrated and purified by flash chromatography (1→3% methanol/dichloromethane) to afford the title compound as a colorless oil. MS 294 (M+H)⁺; ¹H NMR (400 MHz, CD₃OD) δ 7.22 (dd, 1H, J=8.4, 7.6 Hz); 7.18 (d, 1H, J=7.6 Hz); 6.9 (t, 1H, J=7.6); 6.83 (d, 1H, J=8.4 Hz); 4.0 (t, 2H, J=5.8 Hz); 3.75 (bs, 4H); 3.65 (s, 3H); 3.6 (s, 2H); 2.5 (bs, 6H); 1.98 (bs, 2H).

### Preparation V1

[2-(2-morpholin-4-yl-ethoxy)-phenyl]-acetic acid

10% Pd/C (2 g) was added to a solution of [2-(2-morpholin-4-yl-ethoxy)-phenyl]-acetic acid benzyl ester (10 g, 28.2 mmol) in absolute ethanol (100 mL) and was shaken under 40 psi of hydrogen for 4 h. The reaction was purged with nitrogen and filtered. The filtrate was concentrated and the product crystallized from ethanol/ether to afford 7.0 g of the title product as a colorless solid.

MS 266 (M+H)⁺; ¹H NMR (400 MHz, CD₃OD) δ 7.19 (m, 2H); 6.93 (m, 2H); 4.3 (m, 2H); 3.9 (m, 4H); 3.5 (s, 2H); 3.22 (m, 2H); 3.01 (m, 4H).

### Preparation W1

[2-(2-Diethylaminoethoxy)phenyl]acetic acid benzyl ester

Cesium carbonate (1 g, 3.1 mmol) was added to a solution of (2-hydroxy phenyl)acetic acid benzyl ester (300 mg, 1.2 mmol) and N,N-diethyl-2-chloro ethyl amine hydrochloride (200 mg, 1.2 mmol) in dry DMF (10 mL) and the mixture was stirred at RT for 48 h. The mixture was concentrated in vacuo and purified by flash chromatography (12-4% MeOH/CH₂Cl₂) to afford 350 mg of the title compound as a tan oil.

MS 343 (M+H)⁺; ¹H NMR (400 MHz, d6 acetone) δ 7.38 (m, 5H); 7.22 (m, 2H); 6.98 (d, 1H, J=7.9 Hz); 6.88 (t, 1H, J=7.1 Hz); 5.12 (s, 2H); 4.0 (m, 2H); 3.7 (s, 2H); 2.78 (m, 2H); 2.58 (m, 4H); 1.0 (m, 6H).

### Preparation X1

[2-(2-morpholin-4-yl-ethoxy)-phenyl]-acetic acid benzyl ester

Diethylazodicarboxylate (12.3 mL, 77.5 mmol) was added dropwise to a solution of (2-hydroxy-phenyl) acetic acid benzyl ester (15 g, 62 mmol), triphenyl phosphine (20.3 g, 77.5 mmol) and 2-morpholin-4-yl-ethanol (11.25 mL, 92.7 mmol) in dry THF (100 mL) in a cold water bath. The mixture was stirred at RT for 20 h then concentrated to ˜50 mL. The residue was acidified with 1N HCl and washed with ether (6×200 mL). The organic layers were discarded and the aqueous layer was basified to pH 10 with solid Na₂CO₃, and extracted with ether (2×200 mL). The ether layers were combined, dried (MgSO₄), filtered and concentrated. Flash chromatography (5% THF/CH₂Cl₂ containing 0.5% sat NH₃/MeOH) afforded 17 g of the title compound as an oil.

MS 356 (M+H)⁺; ¹H NMR (400 MHz, CDCl₃) δ 7.4-7.2 (m, 7H); 6.92 (t, 1H, J=7.5 Hz); 6.83 (d, 1H, J=7.5 Hz); 5.1 (s, 2H); 4.05 (m, 2H); 3.65 (m, 6H); 2.7 (m, 2H); 2.51 (m, 4H).

The following compound, Preparation X2, was prepared from the appropriate alcohol according to the general procedure above for the preparation of X1.

### Preparation X2

[2-(3-bromo-propoxy)-phenyl]-acetic acid methyl ester

### Preparation Y1

(2-Hydroxy-phenyl)acetic acid benzyl ester

A solution of (2-hydroxy-phenyl)-acetic acid (15 g, 0.1 mol) in dry DMF (120 mL) was treated with benzyl bromide (13 mL, 0.11 mol) and sodium bicarbonate (12.5 g, 0.15 mol) at RT. After stirring for 18 h, an additional 3 mL of benzyl bromide and 5 g of sodium bicarbonate were added and stirring continued for an additional 20 h. The mixture was diluted with water (300 mL) and extracted with ethyl acetate (3×100 mL). The combined organic layers were washed with NaHCO₃ sol (1×) and brine, dried (Na₂SO₄), filtered and concentrated. Upon the addition of hexanes, the product crystallized, was filtered and dried to afford 21 g of the title compound as colorless crystals.

MS 243 (M+H)⁺; ¹H NMR (400 MHz, CDCl₃) δ 7.38 (m, 5H); 7.2 (t, 1H, J=7.1 Hz); 7.08 (d, 1H, J=7.1 Hz); 6.95 (d, 1H, J=7.6Hz); 6.7 (t, 1H, J=7.6 Hz); 5.18 (s, 2H); 3.71 (s, 2H).

### Preparation Z1

trans (2-Allyloxy cyclohexyl)acetic acid

Sodium hydroxide (1 mL of a 15% solution) was added to a solution of (2-allyloxy-cyclohexyl)acetic acid allyl ester (322 mg, 1.37 mmol) in methanol (6 mL) at RT. The mixture was heated at 65° C. for 1.5 h, cooled, and concentrated. The residue was treated with 1N HCl and extracted with EtOAc (2×). The combined extracts were dried (MgSO₄), filtered, and concentrated to afford 262 mg of the title compound as a colorless oil.

MS 199 (M+H)⁺; ¹H NMR (400 MHz, d6 acetone) δ 5.9 (m, 1H); 5.22 (dd, 1H, J=17.5, 2.1 Hz); 5.06 (dd, 1H, J=10.5, 2.1 Hz); 4.1 (dd, 1H, J=12.9, 5.4 Hz); 3.9 (dd, 1H, J=13.2, 5.4 Hz); 3.0 (m, 1H); 2.81 (d, 1H, J=13.2 Hz); 2.77 (dd, 1H, J=15.3, 4.2 Hz); 2.1-1.55 (m, 5H); 1.3-1.0 (m, 4H).

The following compound, Preparation Z2, was prepared from the appropriate ester according to the general procedure for the preparation of Z1.

### Preparation Z2

(trans)-2-(Azido-cyclohexyl)acetic acid

### Preparation AA1

(2-Allyloxy-cyclohexyl)acetic acid allyl ester

Sodium hydride (95%, 273 mg, 10.8 mmol) was added to a solution of trans (2-hydroxy cyclohexyl)acetic acid (814 mg, 5.14 mmol) in dry DMF (25 mL) at 0° C. After 15 min, allyl bromide (1 mL, 11.5 mmol) was added and the reaction was allowed to warm to RT. After 4 h, an additional 0.5 mL of allyl bromide was added to the gelatinous mixture and the reaction stirred overnight. The reaction was quenched by the addition of sat NaHCO₃ sol and extracted with EtOAc (3×). The combined organic layers were washed with water and brine, dried (MgSO₄), filtered and concentrated. The product was purified by flash chromatography (5→10→20% EtOAc/hexanes) to afford 322 mg of the title compound as a colorless oil.

MS 239 (M+H)⁺; ¹H NMR (400 MHz, d6 acetone) δ 5.9 (m, 2H); 5.3 (d, 1H, J=17.5 Hz); 5.2 (m, 2H); 5.03 (d, 1H,. J=10.5 Hz); 4.52 (d, 2H, J=5.3 Hz); 4.0 (dd, 1H, J=13.2, 5.4Hz); 3.84 (dd, 1H, J=13.2, 5.8 Hz); 2.98 (m, 1H); 2.8 (d, 1H, J=13.7 Hz); 2.7 (dd, 1H, J=15.0, 4.5 Hz); 2.2-1.58 (m, 5H); 1.3-1.0 (m, 4H).

### Preparation BB1

(trans)-2-(Azido-cyclohexyl)acetic acid ethyl ester

Sodium azide (993 mg, 7.58 mmol) was added to a solution of cis (2-methanesulfonyloxy-cyclohexyl) acetic acid ethyl ester (668 mg, 2.53 mmol) in dry DMF (15 mL) at RT. The mixture was heated at 97° C. for 16 h, cooled, diluted with water and extracted with EtOAc (2×). The combined organic layers were washed with water (2×) and brine, dried (MgSO₄) filtered and concentrated to afford 488 mg of the title compound as a tan oil.

¹H NMR (400 MHz, d6 acetone) δ 4.08 (q, 2H, J=7.0 Hz); 3.15 (m, 1H); 2.6 (m, 1H); 2.18 (m, 1H); 1.8-1.0 (m, 4H); 1.2 (t, 3H, J=7.0 Hz).

### Preparation CC1

cis (2-methanesulfonyloxy-cyclohexyl)acetic acid ethyl ester

Methane sulfonyl chloride (0.25 mL, 3.2 mmol) was added to a solution of cis (2-hydroxy-cyclohexyl) acetic acid ethyl ester (497 mg, 2.7 mmol), pyridine (650 μL, 8 mmol) and DMAP (489 mg, 4 mmol) in dry CH₂Cl₂ (8 mL) at 0° C. The mixture was allowed to warm to RT and stirred for 20 h. The mixture was diluted with CH₂Cl₂ and washed with 1N HCl (3×) and brine, dried (MgSO₄), filtered and concentrated to afford 668 mg of the title compound as a tan oil.

MS 265 (M+H)⁺; ¹H NMR (400 MHz, d6 acetone) δ 4.9 (m, 1H); 4.04 (q, 2H, J=7.0 Hz); 3.1 (s, 3H); 2.42 (dd, 1H, J=16.2, 7.0 Hz); 2.23 (dd, 1H, J=16.2, 7.4 Hz); 2.1 (m, 2H); 1.7-1.3 (m, 7H); 1.2 (t, 3H, J=7.0 Hz).

### Biological Protocols

The utility of the compounds of Formula (I), the stereoisomers and prodrugs thereof, and the pharmaceutically acceptable salts of the compounds, stereoisomers, and prodrugs, in the treatment or prevention of diseases (such as are detailed herein) in animals, particularly mammals (e.g., humans) may be demonstrated by the activity thereof in conventional assays known to one of ordinary skill in the relevant art, including the in vitro and in vivo assays described below. Such assays also provide a means whereby the activities of the compounds of Formula (I) can be compared with the activities of other known compounds.

1. Phosphodiesterase (PDE) Inhibitory Activity

Preferred PDE compounds suitable for use in accordance with the present invention are potent cGMP PDE9 inhibitors. In vitro PDE inhibitory activities against cyclic guanosine 3′,5′-monophosphate (cGMP) and cyclic adenosine 3′,5′-monophosphate (cAMP) phosphodiesterases are determined by measurement of their IC₅₀ values (the concentration of compound required for 50% inhibition of enzyme activity).

Phosphodiesterase 9 can be generated from full length human recombinant clones transfected into SF9 cells as described in Fisher et al., Journal of Biological Chemistry, 1998, 273, 15559-15564.

Assays are performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE9 inhibitors is investigated by assaying a fixed amount of enzyme in the presence of varying inhibitor concentrations and low substrate, (cGMP in a 3:1 ratio unlabelled to [³H]-labeled at a concentration of about 1/3 Km) such that IC₅₀≅Kᵢ. The final assay volume is made up to 100 μl with assay buffer [20 mM Tris-HCl pH 7.4, 5 mM MgCl₂, 1 mg/ml bovine serum albumin]. Reactions are initiated with enzyme, incubated for 30-60 minutes at 30° C. to give <30% substrate turnover and terminated with 50 μl yttrium silicate SPA beads (containing 3 mM of the respective unlabelled cyclic nucleotide for PDEs 9 and 11). Plates are re-sealed and shaken for 20 minutes, after which the beads are allowed to settle for 30 minutes in the dark and then counted on a TopCount plate reader (Packard, Meriden, Conn.) Radioactivity units are converted to percent activity of an uninhibited control (100%), plotted against inhibitor concentration and inhibitor IC₅₀ values obtained using the ‘Fit Curve’ Microsoft Excel extension.

2. Effect of Specific PDE9 Inhibitors on Metabolic Syndrome in Animals—Effects on Plasma Glucose, Triglyceride, Insulin, and cGMP Levels in ob/ob Mice. Biological Data

a. Test Compounds:

The PDE9 inhibitor compounds to be tested are solubilized in 10% DMSO/0.1% Pluronic® P105 Block Copolymer Surfactant (BASF Corporation, Parsippany, N.J.) in 0.1% saline without pH adjustment and dosed via oral gavage using mouse oral feeding needles (20 gauge, Popper & Sons, Inc., New Hyde Park, N.Y.). A volume of 4 ml/kg weight is administered for each dose. Alternatively, compounds are administered in powdered mouse chow (Mouse Breeder/Auto-JL K20 mouse chow, PMI Feeds, Inc., St. Louis, Mo. that may be custom ground by Research Diets, Inc., New Brunswick, N.J.) as a compound/chow admixture; compounds are mixed with the chow such that the animal will consume specified doses. Compounds are tested at doses ranging from 1-500 mg/kg/day.

b. Experimental Animals:

Male and female ob/ob mice are obtained from Jackson Laboratories (Bar Harbor, Me.) and are used in the studies at 6 to 10 weeks of age. Mice are housed five per cage and allowed free access to Dl1 mouse chow (Purina, Brentwood, Mo.) and water.

c. Experimental Protocol:

Mice are allowed to acclimate to the Pfizer animal facilities for one week prior to the start of the study. When compounds are administered in powdered mouse chow, mice are switched to the powdered diet 3 days prior to the start of the dosing period to allow them to adapt to the change in diet. At this time mice are randomly assigned to groups of ten with five mice per cage. On day one, retro-orbital blood samples are obtained and plasma glucose is determined as described hereinafter. On day one, for mice that are administered compounds as a chow admixture, the chow is replaced with compound-containing chow admixture and this is replenished every other day for the course of the study. On day five, mice are bled from the retro-orbital sinus at approximately 8:00 am for plasma preparation for glucose and triglyceride analysis as described below. Terminal plasma samples are then collected immediately following the retro-orbital sinus bleed as described below. On day one, for compounds that are administered by oral gavage, mice are dosed with vehicle or a test PDE9 inhibitor compound only in the afternoon. Subsequently, mice are dosed twice a day on day 2-4 in the morning and in the afternoon. On day five, the mice receive an a.m. dose and are bled 3 hours later for plasma preparation for glucose and triglyceride analysis as described below. Terminal plasma samples are collected on day five following the retro-orbital sinus bleed as described below. Body weight is measured on days one and five of the study, and food consumption is assessed over the five-day period.

d. Terminal Bleed and Tissue Collection:

On the morning of the last day of the study, mice that are administered the compounds as a chow admixture are bled at approximately 8:00 am via the retro-orbital sinus and then a terminal plasma collection is immediately performed as described hereinafter. On the morning of the last day of the study, mice that are administered compounds via oral gavage are dosed with test compound or vehicle at approximately 8:00 am. Three hours after dosing, 25 μL of blood is obtained via the retro-orbital sinus and is added to 100 μL of 0.025 percent heparinized-saline in Denville Scientific brand microtubes (Denville Scientific Inc., Metuchen, N.J.). The tubes are spun at the highest setting in a Beckman brand Microfuge 12 (Beckman Coulter Inc., Fullerton, Calif.) for 2 minutes. Plasma is collected for plasma glucose and triglyceride determination. The mice are then sacrificed by decapitation and about one milliliter of blood is collected in Becton-Dickinson Microtainer brand plasma separator tubes (Becton-Dickinson Inc., Franklin Lakes, N.J.) with lithium heparin. The tubes are spun in a Beckman Microfuge 12 at the maximum setting for five minutes. Plasma is collected in 1.5 ml Eppendorf tubes and snap frozen in liquid nitrogen. Plasma samples are stored at −80° C. until analyzed.

e. Metabolite and Hormone Analysis:

Plasma glucose, triglycerides, and cholesterol are measured using the Roche/Hitachi 912 Clinical Chemistry Analyzer (Roche Diagnostics Corp., Indianapolis, Ind.) using kits supplied by Roche. Free fatty acids are measured on the same instrument using a kit from Wako Chemical (Richmond, Va.). Plasma cGMP is measured using the Biotrak enzyme-immunoassay system by Amersham (Piscataway, N.J.). Plasma insulin measurements are assessed via a similar technique using the Mercodia ELISA Insulin kit supplied by ALPCO (Uppsala, Sweden). All assays are conducted according to instructions provided by the manufacturers.

f. Results

The PDE9 inhibitor has a greater than 40% inhibition against PDE9 at a concentration of 1 μM. In some compounds, the PDE9 inhibitor has an IC₅₀ of less than 500 nM. In other compounds, the PDE9 inhibitor has an IC₅₀ of less than 50 nM. Is some compounds, the PDE9 inhibitor has a selectivity for PDE9 over PDE1 of greater than 10. In other compounds, PDE inhibitor selectivity for PDE9 over PDE1 is greater than 50, and in still other compounds, greater than 100.

Taken together, our experimental results in a-hyperglycemic, insulin-resistant ob/ob mouse, suggest that selective PDE9 inhibition improves metabolic parameters associated with metabolic syndrome.

We claim:

1. A compound of Formula (I), 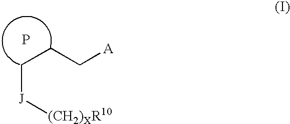
a stereoisomer or prodrug thereof, or a pharmaceutically acceptable salt of said compound, stereoisomer, or prodrug, wherein:
A is 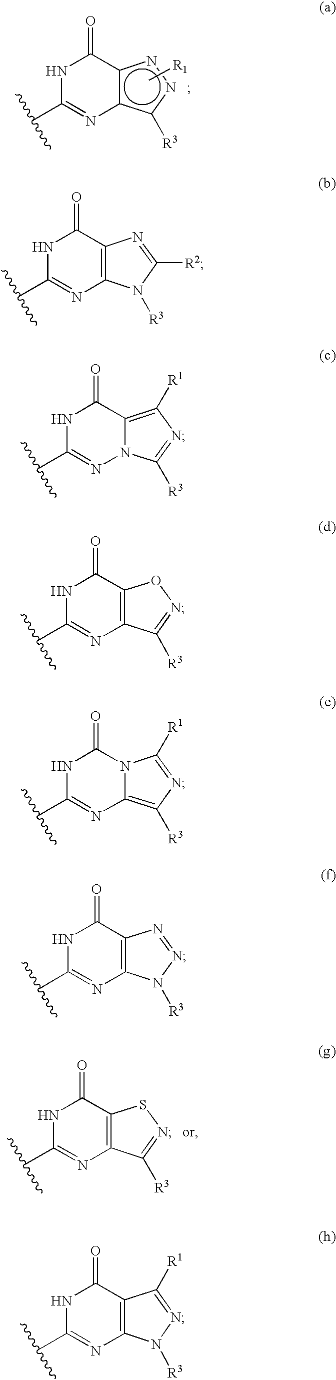
P, including the carbon atoms to which it is attached, is (C₃-C₈)cycloalkyl, (C₃-C₈)heterocycloalkyl, aryl, or heteroaryl; optionally and independently substituted with from 1 to 3 substituents independently selected from halogen, (C₁-C₅)alkyl, (C₁-C₅)alkoxy, and trifluoromethyl;
J is O, S, —N(R¹⁵)—, —N(R¹⁵)CO—, —CON(R¹⁵)—, —SO₂N(R¹⁵)—, or —N(R¹⁵)SO₂—;
x is 0, 1, 2, 3, 4, 5, or 6;
R¹⁰ is —CO₂H, —CONR³⁰R³¹, —NR³⁰R³¹, or —N(R¹⁵)SO₂R⁴⁰;
R¹ and R² are independently H or (C₁-C₃)alkyl;
R³ is (C₁-C₈)alkyl, (C₃-C₈)cycloalkyl, (C₃-C₈)cycloalkyl-methyl, (C₃-C₈)heterocycloalkyl, (C₃-C₈)heterocycloalkyl-methyl, aryl, or heteroaryl; optionally and independently substituted with from 1 to 3 substituents independently selected from halogen, hydroxy, oxo, (C₁-C₅)alkyl, and (C₁-C₅)alkoxy;
R¹⁵ is H or (C₁-C₅)alkyl;
R³⁰ and R³¹ are taken separately and are independently H, (C₁-C₅)alkyl, (C₃-C₈)cycloalkyl, (C₃-C₈)heterocycloalkyl, aryl, or heteroaryl, wherein said R³⁰ and R³¹ are optionally and independently substituted with from 1 to 3 substituents independently selected from halogen, oxo, (C₁-C₅)alkyl, —CO₂R⁴⁰, —COR⁴⁰, —OR⁴⁰, —CONR⁵⁰R⁵¹, —NR⁵⁰R⁵¹, and —SO₂R⁴⁰; or
R³⁰ and R³¹ are taken together with the nitrogen atom to which they are attached to form a 5- to 8-membered heterocycloalkyl ring, said ring optionally having 1 additional heteroatom independently selected from N, O, and S, wherein said 5- to 8-membered heterocycloalkyl ring is optionally and independently substituted with from 1 to 3 substituents independently selected from halogen, oxo, (C₁-C₅)alkyl, —CO₂R⁴⁰, —COR⁴⁰, —OR⁴⁰, —CONR⁵⁰R⁵¹, —NR⁵⁰R⁵¹, and —SO₂R⁴⁰;
R⁴⁰ is H, (C₁-C₅)alkyl, (C₃-C₈)cycloalkyl, (C₃-C₈)heterocycloalkyl, aryl, heteroaryl;
R⁵⁰ and R⁵¹ are taken separately and are independently H, (C₁-C₅)alkyl, (C₃-C₈)cycloalkyl, (C₃-C₈)heterocycloalkyl, aryl, or heteroaryl; or
R⁵⁰ and R⁵¹ are taken together with the nitrogen atom to which they are attached to form a 5- to 8-membered heterocycloalkyl ring, said ring optionally having 1 additional heteroatom independently selected from N, O, and S.

2. A compound of claim 1 wherein:
A is (a), (b), (c), or (h),
R¹ and R² are H;
R³ is (C₃-C₆) alkyl or (C₃-C₅) cycloalkyl;
P is (C₃-C₈)cycloalkyl or aryl;
J is O or S; and,
x is 1, 2, or 3.

3. A compound of claim 2 wherein:
A is (a) or (b).

4. A compound of claim 1 which is:
1-{[2-(3-isopropyl-7-oxo-6,7-dihydro-1H-pyrazolo[4,3-d]pyrimidin-5-ylmethyl)-phenoxy]-acetyl}-pyrrolidine-2-carboxylic acid;
1-{[2-(1-cyclopentyl-4-oxo-4,5-dihydro-1H-pyrazolo[3,4-d]pyrimidin-6-ylmethyl)-phenoxy]-acetyl}-pyrrolidine-2(S)-carboxylic acid
3-isopropyl-5-[2-(2-oxo-2-piperazin-1-yl-ethoxy)-benzyl]-1,6-dihydro-pyrazolo[4,3-d]pyrimidin-7-one;
1-cyclopentyl-6-[2-(2-oxo-2-piperazin-1-yl-ethoxy)-benzyl]-1,5-dihydro-pyrazolo[3,4-d]pyrimidin-4-one
3-isopropyl-5-[2-(2-morpholin-4-yl-2-oxo-ethoxy)-benzyl]-1,6-dihydro-pyrazolo[4,3-d]pyrimidin-7-one;
3-isopropyl-5-[2-(2-oxo-2-pyrrolidin-1-yl-ethoxy)-benzyl]-1,6-dihydro-pyrazolo[4,3-d]pyrimidin-7-one;
5-{2-[2-(4-ethyl-piperazin-1-yl)-2-oxo-ethoxy]-benzyl}-3-isopropyl-1,6-dihydro-pyrazolo[4,3-d]pyrimidin-7-one;
N,N-diethyl-2-[2-(3-isopropyl-7-oxo-6,7-dihydro-1H-pyrazolo[4,3-d]pyrimidin-5-ylmethyl)-phenoxy]-acetamide;
1-{[2-(3-isopropyl-7-oxo-6,7-dihydro-1H-pyrazolo[4,3-d]pyrimidin-5-ylmethyl)-phenoxy]-acetyl}-pyrrolidine-2-carboxylic acid methyl ester;
4-{[2-(3-isopropyl-7-oxo-6,7-dihydro-1H-pyrazolo[4,3-d]pyrimidin-5-ylmethyl)-phenoxy]-acetyl}-piperazine-1-carboxylic acid tert-butyl ester;
N-(2-dimethylamino-ethyl)-2-[2-(3-isopropyl-7-oxo-6,7-dihydro-1H-pyrazolo[4,3-d]pyrimidin-5-ylmethyl)-phenoxy]-acetamide;
1-{[2-(1-cyclopentyl-4-oxo-4,5-dihydro-1H-pyrazolo[3,4-d]pyrimidin-6-ylmethyl)-phenoxy]-acetyl}-pyrrolidine-2-carboxylic acid methyl ester;
4-{[2-(1-cyclopentyl-4-oxo-4,5-dihydro-1H-pyrazolo[3,4-d]pyrimidin-6-ylmethyl)-phenoxy]-acetyl}-piperazine-1-carboxylic acid tert-butyl ester;
1-cyclopentyl-6-[2-(2-oxo-2-pyrrolidin-1-yl-ethoxy)-benzyl]-1,5-dihydro-pyrazolo[3,4-d]pyrimidin-4-one;
1-cyclopentyl-6-[2-(2-morpholin-4-yl-2-oxo-ethoxy)-benzyl]-1,5-dihydro-pyrazolo[3,4-d]pyrimidin-4-one;
2-[2-(1-cyclopentyl-4-oxo-4,5-dihydro-1H-pyrazolo[3,4-d]pyrimidin-6-ylmethyl)-phenoxy]-N-(2-dimethylamino-ethyl)-acetamide;
1-cyclopentyl-6-{2-[2-(4-ethyl-piperazin-1-yl)-2-oxo-ethoxy]-benzyl}-1,5-dihydro-pyrazolo[3,4-d]pyrimidin-4-one;
2-[2-(1-cyclopentyl-4-oxo-4,5-dihydro-1H-pyrazolo[3,4-d]pyrimidin-6-ylmethyl)-phenoxy]-N,N-diethyl-acetamide;
[2-(3-isopropyl-7-oxo-6,7-dihydro-1H-pyrazolo[4,3-d]pyrimidin-5-ylmethyl)-phenoxy]-acetic acid;
[2-(1-cyclopentyl-4-oxo-4,5-dihydro-1H-pyrazolo[3,4-d]pyrimidin-6-ylmethyl)-phenoxy]-acetic acid;
3-isopropyl-5-[2-(5-chloro-2-morpholin-4-yl-ethoxy)-benzyl]-1,6-dihydro-pyrazolo[4,3-d]pyrimidin-7-one;
3-isopropyl-5-[2-(2-pyrrolidin-1-yl-ethoxy)-benzyl]-1,6-dihydro-pyrazolo[4,3-d]pyrimidin-7-one;
3-isopropyl-5-[2-(2-morpholin-4-yl-ethoxy)-cyclohexylmethyl]-1,6-dihydro-pyrazolo[4,3-d]pyrimidin-7-one;
5-[5-fluoro-2-(2-morpholin-4-yl-ethoxy)-benzyl]-3-isopropyl-1,6-dihydro-pyrazolo[4,3-d]pyrimidin-7-one;
3-cyclopentyl-5-[5-fluoro-2-(2-morpholin-4-yl-ethoxy)-benzyl]-1,6-dihydro-pyrazolo[4,3-d]pyrimidin-7-one;
3-isopropyl-5-[2-(2-morpholin-4-yl-ethoxy)-benzyl]-1,6-dihydro-pyrazolo[4,3-d]pyrimidin-7-one;
9-(1,2-dimethyl-propyl)-2-[2-(2-morpholin-4-yl-ethoxy)-benzyl]-1,9-dihydro-purin-6-one;
2-[2-(2-morpholin-4-yl-ethoxy)-benzyl]-9-(tetrahydro-furan-3-yl)-1,9-dihydro-purin-6-one;
5-[2-(2-diethylamino-ethoxy)-benzyl]-3-isopropyl-1,6-dihydro-pyrazolo[4,3-d]pyrimidin-7-one;
3-cyclopentyl-5-[2-(2-morpholin-4-yl-ethoxy)-benzyl]-1,6-dihydro-pyrazolo[4,3-d]pyrimidin-7-one;
3-cyclobutyl-5-[2-(2-morpholin-4-yl-ethoxy)-benzyl]-1,6-dihydro-pyrazolo[4,3-d]pyrimidin-7-one;
9-(1(R),2-dimethyl propyl)-[2-(2-morpholin-4-yl-ethoxy)-benzyl]-1,9-dihydro-purin-6-one;
9-(2-methyl-butyl)-2-[2-(2-morpholin-4-yl-ethoxy)-benzyl]-1,9-dihydro-purin-6-one;
9-cyclopentyl-2-[2-(2-morpholin-4-yl-ethoxy)-benzyl]-1,9-dihydro-purin-6-one;
5-[2-(2-morpholin-4-yl-ethoxy)-benzyl]-3-pyridin-3-yl-1,6-dihydro-pyrazolo[4,3-d]pyrimidin-7-one;
9-(1,2-dimethyl-propyl)-2-[2-(2-morpholin-4-yl-ethoxy)-benzyl]-1,9-dihydro-purin-6-one;
9-isopropyl-2-[2-(2-morpholin-4-yl-ethoxy)-benzyl]-1,9-dihydro-purin-6-one;
2-[2-(2-morpholin-4-yl-ethoxy)-benzyl]-9-(tetrahydro-furan-2-ylmethyl)-1,9-dihydro-purin-6-one;
9-(1-isopropyl-2-methyl-propyl)-2-[2-(2-morpholin-4-yl-ethoxy)-benzyl]-1,9-dihydro-purin-6-one;
9-(1-ethyl-propyl)-2-[2-(2-morpholin-4-yl-ethoxy)-benzyl]-1,9-dihydro-purin-6-one;
9-cyclopentyl-8-methyl-2-[2-(2-morpholin-4-yl-ethoxy)-benzyl]-1,9-dihydro-purin-6-one;
3-cyclopentyl-5-[2-(2-morpholin-4-yl-ethoxy)-benzyl]-3,6-dihydro-[1,2,3]triazolo[4,5-d]pyrimidin-7-one;
1-cyclopentyl-6-[2-(2-morpholin-4-yl-ethoxy)-benzyl]-1,5-dihydro-pyrazolo[3,4-d]pyrimidin-4-one;
9-cyclopentyl-2-[2-(3-morpholin-4-yl-propoxy)-benzyl]-1,9-dihydro-purin-6-one;
N-[(1R,2S)2-(3-isopropyl-7-oxo-6,7-dihydro-1H-pyrazolo[4,3-d]pyrimidin-5-ylmethyl)-cyclohex-1-yl]-2-pyrrolidin-1-yl-acetamide;
N-[(1R,2S)2-(3-isopropyl-7-oxo-6,7-dihydro-1H-pyrazolo[4,3-d]pyrimidin-5-ylmethyl)-cyclohex-1-yl]-2-morpholin-4-yl-acetamide;
2-diethylamino-N-[(1R,2S)2-(3-isopropyl-7-oxo-6,7-dihydro-1H-pyrazolo[4,3-d]pyrimidin-5-ylmethyl)-cyclohex-1-yl]-acetamide; 1-{[(1R,2S)2-(3-isopropyl-7-oxo-6,7-dihydro-1H-pyrazolo[4,3-d]pyrimidin-5-ylmethyl)-cyclohex-1-ylcarbamoyl]-methyl}-pyrrolidine-2(S)-carboxylic acid methyl ester;
2-cyclobutylamino-N-[(1R,2S)2-(3-isopropyl-7-oxo-6,7-dihydro-1H-pyrazolo[4,3-d]pyrimidin-5-ylmethyl)-cyclohex-1-yl]-acetamide; or
2-cyclopropylamino-N-[(1R,2S)2-(3-isopropyl-7-oxo-6,7-dihydro-1H-pyrazolo[4,3-d]pyrimidin-5-ylmethyl)-cyclohex-1-yl]-acetamide;
a stereoisomer or prodrug thereof, or a pharmaceutically acceptable salt of said compound, stereoisomer, or prodrug.

5. A pharmaceutical composition comprising a compound of claim 1, a stereoisomer or prodrug thereof, or a pharmaceutically acceptable salt of said compound, stereoisomer, or prodrug; and a pharmaceutically acceptable carrier, vehicle, or diluent.

6. A method of treating a condition, disease, or symptom selected from the group consisting of type 1 diabetes, type 2 diabetes, hyperglycemia, dyslipidemia, impaired glucose tolerance, metabolic syndrome, and cardiovascular disease, wherein said method comprises administering to a mammal in need of such treatment or prevention, a therapeutically effective amount of a compound of claim 1, a stereoisomer or prodrug thereof, or a pharmaceutically acceptable salt of said compound, stereoisomer, or prodrug; or a pharmaceutical composition comprising said compound of claim 1, or said stereoisomer or prodrug thereof, or said pharmaceutically acceptable salt of said compound, stereoisomer, or prodrug.

7. A method of claim 6 wherein said condition, disease, or symptom is diabetes or cardiovascular disease.

8. A method of inhibiting phosphodiesterase 9 activity in a mammal in need of such inhibition which method comprises administering a phosphodiesterase 9 inhibiting amount of a compound of claim 1, a stereoisomer or prodrug thereof, or a pharmaceutically acceptable salt of said compound, stereoisomer, or prodrug; or a pharmaceutical composition comprising a compound of Formula (I), a stereoisomer or prodrug thereof, or a pharmaceutically acceptable salt of said compound, stereoisomer, or prodrug, and a pharmaceutically acceptable carrier, vehicle, or diluent.
